(19) United States
(12) Patent Application Publication
(10) Pub. No.: US 2003/0082129 A1
(43) Pub. Date: May 1, 2003

(54) Hair and skin care compositions containing siloxane-based polyamide copolymers

(76) Inventors: Anne Marie Buckingham, Midland, MI (US); Regina Marie Malczewski, Midland, MI (US); Deborah Lynn Meyers, Midland, MI (US); Benedicte Courel, Buizingen (BE); Sabrian Marchioretto, Bruxelles (BE); Adriana Urrutia-Gutierrez, Col.Mixcoac (MX)

Correspondence Address: Alan Zombeck, DOW CORNING CORPORATION CO 1232, 2200 W. SALZBURG ROAD, P.O. BOX 994, MIDLAND, MI, 48686-0994, US

(21) Appl. No.: 10/213,906

(22) Filed: Aug. 6, 2002

Related U.S. Application Data
(60) Provisional application No. 60/310,613, filed on Aug. 7, 2001.

(51) Int. Cl. A61K 7/06; A61K 7/11

(52) U.S. Cl. 424/70.12; 424/70.14

(57) ABSTRACT

Hair and skin care compositions containing siloxane-based polyamides are disclosed, some of which utilize a base composition comprising a silicone fluid, a silicone immiscible substance, and a siloxane-based polyamide. The siloxane-based polyamide functions as a compatibilizing agent, film forming agent and durability enhancer in these hair and skin care compositions. A method of enhancing the fragrance longevity of fragrance containing skin care compositions after application is also disclosed.

### CROSS-REFERENCE

This application is related to and claims priority of U.S. Provisional Patent Application Serial No. 60/310,613, filed Aug. 7, 2001.

### FIELD OF THE INVENTION

This invention provides certain hair and skin care compositions containing siloxane-based polyamides, some of which utilize a base composition comprising a silicone fluid, a silicone immiscible substance, and a siloxane-based polyamide. The siloxane-based polyamide functions as a compatibilizing agent, film forming agent and durability enhancer in the hair and skin care compositions of the present invention. The present invention also provides a method of enhancing the fragrance longevity of fragrance containing skin care compositions after application.

### BACKGROUND OF THE INVENTION

The trend in the hair and skin care market is for the consumer to spend less time on personal hygiene, yet require better performing products. As a result, a need exists for hair and skin care products with multiple benefits. Examples in hair care include: shampoos that provide cleaning, conditioning, and light style retention; hair conditioners that provide conditioning and light style retention; and hair styling products that provide flexible hold and soft feel. Examples in skin care include the need for moisturizers, sunscreens, and color cosmetics products to provide additional consumer perceived benefits such as enhanced aesthetics and longer lasting performance.

These needs have been the impetus behind the development of new raw materials used to formulate these consumer products. One general class of materials that have been used for this purpose has been silicone-organic block and graft copolymers. These copolymers combine the properties of organics, such as solubility and strength, with the elastic properties of silicones. Representative examples of this art can be found in U.S. Pat. Nos. 5,120,531, 5,500,209, 5,243,010, 5,272,241, and WO97/36572.

One particular silicone-organic copolymer that has recently been disclosed is siloxane-based polyamide. For example, U.S. Pat. Nos. 6,051,216 and 6,353,076 B1 disclose siloxane-based polyamides that can be used as gelling agents to thicken cosmetic compositions. Furthermore, U.S. Pat. No. 5,981,680 discloses an improved method for making siloxane-based polyamides. While these references teach the use of siloxane-based polyamides as thickening agents in cosmetic compositions, they do not disclose certain skin and hair care compositions containing siloxane-based polyamides.

It is an object of the present invention to provide hair care compositions containing siloxane-based polyamides having improved styling and/or hold properties, protection of hair color, improved aesthetics and longer lasting fragrance release.

It is another object of the present invention to provide skin care and topical pharmaceutical compositions containing siloxane-based polyamides useful for delivering a wide variety of cosmetic materials and pharmaceutical actives to the skin, having improved aesthetics, improved non transfer properties, and longer lasting fragrance release.

### SUMMARY OF THE INVENTION

The foregoing objectives are achieved by the present invention by using siloxane-based polyamides as compatibilizing agents, film forming agents and durability enhancers to formulate hair and skin care compositions.

The present invention provides a base composition comprising;

A) a silicone fluid,

B) a silicone immiscible organic substance,

C) a siloxane-based polyamide,

wherein the base composition is a stable dispersion. The present inventors have discovered that siloxane-based polyamides can be used to compatibilize a silicone fluid with a silicone immiscible organic substance. This base composition can be used in various hair and skin care compositions.

The present invention also relates to hair care compositions containing siloxane-based polyamides which leave the hair feeling natural, i.e. not very stiff or sticky, retain hair color, provide improved shine, and longer lasting fragrance.

The present invention further relates to skin care compositions containing siloxane-based polyamides. The siloxane-based polyamides provide skin care compositions that easily spread on skin to provide a highly aesthetic, substantive and long lasting film. Furthermore, the present inventors have found certain siloxane-based polyamides provide skin care compositions that are useful for delivering a wide variety of fragrance, cosmetic, and pharmaceutical actives to the skin. The film forming and barrier properties of these siloxane-based polyamides provide compositions that give durability to cosmetic and pharmaceutical actives. In particular, the present inventors have identified certain siloxane-based polyamides provide for longer lasting fragrance after application of the skin care compositions containing such. Thus, the present invention also provides a method of enhancing the fragrance longevity of fragrance containing skin care compositions after application.

### Definitions and Usage of Terms

The following is a list of definitions, as used herein.

“A” and “an” each mean one or more.

“Alkyl” means a saturated hydrocarbon group.

“Carrier” a general description of the type of composition used in a personal care formulation. This term is synonymous with “vehicle” as used in the art for describing how a personal care active ingredient is delivered to the hair or skin by the formulation. Some examples of carriers include; emulsions, solvents, dispersions, and suspensions.

“Clear solution” as used herein refers to a solution having a 90% or greater transmittance of visible light. Transmittance is defined by the ratio of the intensity of light emerging from a certain thickness of the formulation to the intensity of light emerging from equal thickness of the pure solvent, which is water, multiplied by 100.

“Combination” means two or more items put together by any method.

“Copolymer” means a polymer made from at least two distinct monomers. Copolymer includes, but is not limited to, polymers made from only two distinct monomers.

CTFA—refers to the Cosmetic, Toiletries, & Fragrance Association

“Immiscible” means inability of one liquid to dissolve uniformly another liquid.

“Miscible” means the ability of one liquid to disperse uniformly another liquid.

“Stable dispersion” as used herein refers to a dispersion that does not show physical signs of separation upon standing for 24 hours.

All amounts, ratios, and percentages are by weight unless otherwise indicated.

### DETAILED DESCRIPTION OF THE INVENTION

The siloxane-based polyamides useful as components in the skin and hair care compositions of the present invention comprises a unit represented by the following Formula A:

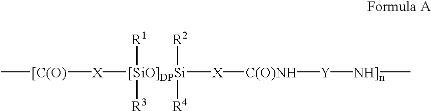

where:

(1) DP (degree of polymerization) is 1-700, alternatively 10-500, or alternatively 15-100. DP represents an average value for degree of polymerization of the siloxane units in the polymer with greater or lesser DP values centered around the indicated DP value.

(2) n is 1-500, alternatively 1-100, or alternatively 4-25.

(3) X is a linear or branched chain alkylene having 1-30 carbons, typically 3-10 carbons, or alternatively 10 carbons.

(4) Y is a linear or branched chain alkylene having 1-40 carbons, alternatively 1-20 carbons, alternatively 2-6 carbons, or alternatively 6 carbons wherein

(a) the alkylene group may optionally and additionally contain in the alkylene portion at least one of (i) 1-3 amide linkages; (ii) a C5 or C6 cycloalkane; or (iii) phenylene, optionally substituted by 1-3 members which are independently C1-C3 alkyls; and

(b) the alkylene group itself may optionally be substituted by at least one of (i) hydroxy; (ii) a C3-C₈ cycloalkane; (iii) 1-3 members which are independently C1-C3 alkyls; phenyl, optionally substituted by 1-3 members which are independently C1-C3 alkyls; (iv) a C1-C3 alkyl hydroxy; or (v) a C1-C6 alkyl amine; and

(c) Y can be Z where Z is T(R²⁰)(R²¹)(R²²) where R²⁰, R²¹ and R²² are each independently linear or branched C1-C10 alkylenes; and T is CR in which R is hydrogen, the group defined for R¹-R⁴, or a trivalent atom such as N, P and Al.

(5) each of R¹-R⁴ (collectively “R”) is independently methyl, ethyl, propyl, isopropyl, a siloxane chain, or phenyl, wherein the phenyl may optionally be substituted by 1-3 members, which are methyl or ethyl. More particularly, R¹-R⁴ are methyl or ethyl, especially methyl.

(6) X, Y, DP, and R¹-R⁴ may be the same or different for each polyamide unit.

Also, the polyamides must have a siloxane portion in the backbone and optionally may have a siloxane portion in a pendant or branched portion.

Representative examples of siloxane-based polyamides useful in the skin and hair care compositions of the present invention are disclosed in U.S. Pat. No. 6,051,216 and U.S. Pat. No. 6,353,076 B1, which are both hereby incorporated by reference.

In the hair and skin care compositions of the present invention, the silicone-polyamide copolymer can be used in an amount of 0.1-80 percent by weight, alternatively 1-30 percent by weight or alternatively 2-20 percent by weight. Typically, the silicone-polyamide copolymer does not exceed 50 percent by weight of the hair or skin composition.

The siloxane-based polyamide can be used directly in various hair and skin care compositions. However, typically the siloxane-based polyamide is first combined with a solvent and optionally a personal care or pharmaceutical active ingredient to form a base composition. The solvent can be either a silicone or organic solvent, acceptable for hair and skin care formulations and the siloxane-based polyamide. Illustrative, non limiting examples include; esters (for example, isopropyl myristate and C12-15 alkyl lactate), silicone fluids (for example, cyclomethicone, dimethicone), low molecular weigth alcohols like ethanol and isopropanol, guerbet alcohols having 8-30 carbons, particularly 12-22 carbons (for example, isolauryl alcohol, isocetyl alcohol, isostearyl alcohol), fatty alcohols (for example, stearyl alcohol, myristyl alcohol, oleyl alcohol), and ethoxylated and propoxylated alcohols (for example, the polyethylene glycol ether of lauryl alcohol that conforms to the formula CH₃(CH₂)₁₀CH₂(OCH₂CH₂)_{b} OH where b has an average value of 4 (also called laureth-4). PPG-14 butyl ether, where the “PPG-14” portion is the polymer of propylene oxide that conforms generally to the formula H(OCH₂C(CH₃)H)_{c}OH, where c has an average value of 14, or PPG-3 myristyl ether which is the polypropylene glycol ether of myristyl alcohol that conforms to the formula CH₃(CH₂)₁₂CH₂(OCH(CH₃)CH₂)_{d}OH where d has an average value of 3. Mixtures of solvents can also be used. Typically, the siloxane-based polyamides are soluble in the solvent system, at least at elevated temperatures, as described in U.S. Pat. No. 5,500,209. The personal care ingredients are described infra. Base compositions useful in the hair and skin care compositions of the present invention are described in U.S. Pat. No. 6,051,216, which is hereby incorporated by reference. While the base compositions described in U.S. Pat. No. 6,051,216 require the use of a silicone miscible solvent, the present inventors have discovered additional base compositions useful in the hair and skin compositions herein comprising;

A) a silicone fluid

B) a silicone immiscible organic substance,

C) a siloxane-based polyamide described above as Formula A,

wherein the composition is a stable dispersion. While U.S. Pat. No. 6,051,216 teaches the use of siloxane-based polyamides to primarily thicken or gel silicone fluids, the present inventors have discovered that siloxane-based polyamides can be used to compatibilize a silicone fluid with a silicone immiscible organic substance.

The silicone fluid or the silicone immiscible organic substance is used in the range of 0.1-90 percent by weight in the base composition of the present invention, alternatively 0.5-70 percent by weight, or alternatively 1-50 percent by weight, with the proviso that the total of these components is 100%. Optionally, additional silicone miscible solvents, mixtures of solvents or personal care ingredients, all described infra, may be added to the base composition.

The silicone fluids useful as component A) in the base composition of the present invention are those known in the art for use in personal care formulations and include volatile and non-volatile silicone fluids, and mixtures of both. Typically, the silicone fluid is a cyclic or linear polydimethylsiloxane. Representative, non-limiting examples of these silicone fluids include those known according to the CTFA designation as cyclomethicone and dimethicone fluids. Illustrative examples of volatile silicones, or volatile methyl siloxanes (VMS) can be found in U.S. Pat. No. 5,880,210, which is hereby incorporated by reference. When a VMS silicone fluid is used, cyclopentasiloxane is typically selected. Alternatively, the silicone fluid can be any polyorganosiloxane having the following general formula; [RₘSi(O)_{4-m/2}]ₙ, where R is independently any monovalent hydrocarbon group and m=1-3, and n is at least two. Typically, the polyorganosiloxane has a viscosity in the range of 0.65 to 50,000 cs (mm/s²). However, if the polyorganosiloxane should have a higher viscosity, or if the polyorganosiloxane is a solid at room temperature, it can be dispersed in a lower viscosity silicone fluid, such as a VMS fluid, to create a silicone fluid suitable in the compositions of the present invention. Alternatively, the silicone fluid is a linear polydimethylsiloxane polymer having a viscosity in the range of 0.65 to 50,000 cs (mm/s²). Alternatively, the silicone fluid can be a phenyl containing polyorganosiloxane, such as those designated as phenyl trimethicone by the CTFA. Illustrative, non-limiting examples of phenyl containing polyorganosiloxanes include DC 554, DC 555, and DC 556 fluids (Dow Corning Corporation, Midland Mich.). The silicone fluid can also be a mixture of any of the silicone fluids described supra.

The silicone immiscible organic substance, that is component B) in the base compositions of the present invention, is selected from either organic oils immiscible with the silicone fluids (component A) of the base composition, or selected from personal care ingredients that are immiscible with the silicone fluids of the base composition.

The silicone immiscible organic oils useful as component B) in the base composition of the present invention include: esters such as C12-15 Alcohol Benzoate, Caprilic/Capric Triglyceride, Caprilic/Capric Triglyceride, Coco-Caprylate/Caprate, Diisopropyl Adipate, Diisostearyl Fumarate, Diisostearyl Malate, Dioctyl Maleate, Isocetyl Stearate, Isopropyl Isostearate, Isopropyl Laurate, Isopropyl Myristate, Isopropyl Myristate, Isopropyl Palmitate, Isopropyl Palmitate, Isopropyl Stearate, Isostearyl Benzoate, Myristyl Ether Acetate, Myristyl Lactate, Octyldodecyl Stearoyl Stearate, Octylpalmitate, Octylpalmitate, Octylstearate, Tridecyl Neopentanoate, and Triisocetyl Citrate; fatty alcohols such as Lauryl Alcohol, Octyldodecanol, Oleyl Alcohol; glycerides such as Glyceryl Trioctanate and Polyglyceryl-3 Diisostearate; hydrocarbons such as Mineral Oil, and Polydecene; vegetable oils such as Almond Oil, Castor Oil, Coconut Oil, Corn Oil, Jojoba Oil, Lanolin Oil, Palm Oil, Sesame Oil, Soybean Oil, and Sunflower Oil.

The silicone immiscible organic substance, component B) in the base compositions of the present invention, can also be a personal care ingredient selected from the personal care ingredients known in the art, with the proviso that the personal care ingredient is immiscible with the selected silicone fluid.

In the base composition of the present invention, the siloxane-based polyamide compatiblizes the selected silicone fluid and silicone immiscible organic substance. One skilled in the art can easily determine if any combination of silicone fluid and organic substance is miscible, or immiscible, by simply mixing the two components at various weight ratios and allowing the mixture to stand for 24 hours and assessing if the mixture is homogenous (miscible) or non-homogenous (immiscible).

Those skilled in the art recognize there are many combinations of silicone fluids and organic substances that are immiscible. In particular, silicone fluids such as linear polydimethylsiloxanes having a viscosity greater than 20 cs (mm/s²) are not compatible with many organic substances that are desirable as ingredients in personal care compositions. In other examples, certain common personal care ingredients, like castor oil, are difficult to compatibilize in a silicone fluid. Thus, in one embodiment of the base composition of the present invention, the silicone fluid is selected from a linear polydimethylsiloxanes having a viscosity equal to or greater than 20 cs (mm/s²). In another embodiment of the base compositions of the present invention, the silicone fluid, silicone immiscible organic, and siloxane-based polyamide are be selected and combined at certain ratios so as to provide clear solutions.

The base composition of the present invention can further comprise a miscible solvent. The miscible solvent can be any solvent known to those skilled in the art for formulating personal care compositions, representative examples of which can be found in U.S. Pat. No. 6,051,216. Alternatively, the miscible solvent can be the solvent described supra for solubilizing the siloxane-based polyamide. The amount of miscible solvent combined with the base composition can vary from 0.05 to 90 weight percent of the entire composition, alternatively, 0.1 to 70 weight percent, or alternatively 0.1 to 50 weight percent.

The base composition of the present invention can further comprise a silicone elastomer. In particular, the silicone elastomers and related compositions described in U.S. Pat. Nos. 5,654,362; 5,811,487; 5,880,210; 6,207,717; 6,200,581; 6,168,782; and 6,221,979 can be combined with the base compositions of the present invention. Representative commercial products of such silicone elastomers include DC 9010, DC 9011, and DC 9040 (Dow Corning Corporation, Midland, Mich.). The amount of silicone elastomer combined with the base composition can vary from 0.05 to 90 weight percent of the entire composition, alternatively, 0.1 to 70 weight percent, or alternatively 0.1 to 50 weight percent.

The base composition of the present invention can further comprise a fragrance, described infra. Typically in these compositions the siloxane-based polyamide as described in formula A above has the following structure, R1, R2, R3 and R4 are methyl, X is (CH₂)₁₀ and Y is (CH₂)₆ and DP is 30 or greater, alternatively, 50 or greater, or alternatively 100 or greater.

The base compositions according to the present invention can easily be prepared by methods known to those skilled in the art, such as by using known mixing procedures.

The present inventors have discovered the siloxane-based polyamides as described above are useful in hair care compositions because these polyamides melt above room temperature. Without being limited by theory, it is believed that when these polyamides are subjected to temperatures above these melting points, they are capable of flowing and can provide flexibility during the styling process (e.g., when curling irons, blow driers, and other heat sources are applied to the hair). Upon cooling of the polyamide copolymer to room temperature, the copolymer is typically below the melting temperature and the copolymer possesses structural rigidity, and yet has flexibility from the polysiloxane blocks, and can provide flexible hair hold or style retention. Additionally, the siloxane blocks of the copolymers provide a smooth silky, feel and shine to the hair.

Furthermore, at skin temperatures, these polyamide copolymers would be at a temperature that is essentially below their melt temperature. The copolymers enhance the film forming properties of skin care compositions, and provide skin feel sensory benefits such as better and more even distribution upon the skin, faster absorbance of the composition, and protection of active ingredients from easily rubbing or washing off the skin.

The present inventors have also discovered the hair and skin care formulations containing fragrances and the siloxane-based polyamides provide formulations that after application to the hair or skin, provide a longer lasting fragrance. In other words, consumers are able to detect the fragrance for a longer period of time as compared to a similar formulation not containing the siloxane-based polyamide copolymer. Not to be limited to any theory, the present inventors believe that the siloxane-based polyamides compatiblized with most fragrance components and because of their film forming properties, act as a barrier to fragrance release in the final personal care formulation after the volatile components have evaporated, thus providing a longer lasting fragrance delivery.

The hair and skin care compositions according to the present invention can easily be manufactured by methods known to those skilled in the art. Typically, the compositions can be made by mixing the various components at an elevated temperature (that is, by heating and mixing the various components) and then cooling in order to form a thickened composition such as a cream or lotion. For cosmetic compositions, additional ingredients can be added using techniques and manufacturing processes known in the art. Typically, any volatile components (such as fragrances) are added to the mixture at a relatively late stage of the mixing, so as to limit volatilization of the volatile components.

The siloxane-based polyamide can be dissolved in a silicone fluid, for example, at elevated temperatures (for example, up to 160 degrees C.) so as to form a solution. It is preferred that the solution is not heated too long or at too high a temperature, so as not to cause the solution to color. The hair or skin care active can be added to the solution of silicone fluid and silicone-polyamide copolymer and mixed to homogeneity. For example, the silicone fluids, other organic solvents and siloxane-based polyamides can be mixed at elevated temperatures so as to dissolve the polymer in the silicone fluid, with cosmetically active ingredients being added to the mixture of silicone fluid and polymer. In the case where an aqueous phase is included, an emulsion is the result. Alternately, as when formulating a solid cosmetic such as a lipstick, the siloxane-based polyamide can be heated above its melting point and added to a hot lipstick base with appropriate mixing. Where the product is a cream product, the molten product, at elevated temperatures, can be poured into dispensing containers and allowed to cool and thicken therein.

The siloxane-based polyamides can be formulated into a wide variety of product types, including mousses, gels, lotions, creams, tonics, sprays, shampoos, sunscreens, anti-acne preparations, topical analgesics, mascaras, lipsticks, color foundations, and the like. The carriers and additional components required to formulate such products vary with product type and can be routinely chosen by one skilled in the art. The following is a description of some of these carriers and additional components.

Carriers

The hair care compositions of the present invention can comprise a carrier, or a mixture of such carriers, which are suitable for application to the hair. The carriers are present at from about 0.5% to about 99.5%, preferably from about 5.0% to about 99.5%, more preferably from about 10.0% to about 98.0%, of the composition. As used herein, the phrase “suitable for application to hair” means that the carrier does not damage or negatively affect the aesthetics of hair or cause irritation to the underlying skin.

Where the hair care compositions are conditioners and rinses, the carrier can include a wide variety of conditioning materials, such as hydrocarbons, silicone fluids, and cationic materials. Where the hair care compositions are shampoos, the carrier can include surfactants, suspending agents, thickeners etc. Various additional components useful in hair care compositions are described in U.S. Pat. No. 4,387,090, to Bolich, Jr. issued Jun. 7, 1983; which are incorporated by reference herein. Some of these additional components are described below.

The topical skin care compositions of the present invention can comprise a carrier. The carrier should be “cosmetically and/or pharmaceutically acceptable”, which means that the carrier is suitable for topical application to the skin, has good aesthetic properties, is compatible with the silicone-polyamide copolymers of the present invention and any other components, and will not cause any untoward safety or toxicity concerns. The carrier can be in a wide variety of forms. For example the carrier can be an emulsion, including but not limited to: conventional oil-in-water and water-in-oil emulsions; as well as so-called multiple phase or triple emulsion such as; water-in-oil-in-water and oil-in-water-in-silicone emulsions. Representative examples of multiple phase emulsions suitable as carriers in the present invention include those found in U.S. Pat. Nos. 5,948,855; 6,238,657; 6,080,394; and 6,410,038, which are hereby incorporated by reference. Other suitable topical carriers include: anhydrous liquid solvents such as oils, alcohols, and silicones (e.g., mineral oil, ethanol, isopropanol, dimethicone, cyclomethicone, and the like); aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following four references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987). Typical cosmetically and/or pharmaceutically acceptable topical carriers include hydro-alcoholic systems and oil-in-water emulsions. When the carrier is an oil-in-water emulsion, the carrier can include any of the common incipient ingredients for preparing these emulsions. Additional components useful in formulating these topical compositions are further described below.

Hair Care Agents

The hair styling polymers (as identified by their CTFA name) useful as a hair care agent in the compositions of the present invention include: Acrylamide/Ammonium Acrylate Copolymer, Acrylamide/Sodium Acrylate Copolymer, Acrylates/Acrylamide Copolymer, Acrylates/Ammonium Methacrylate Copolymer, Acrylates/t-Butylacrylamide Copolymer, Acrylates Copolymer, Acrylates/Dimethicone Copolymer, Acrylates/Dimethylaminoethyl Methacrylate Copolymer, Acrylates/Ethylhexyl Acrylate Crosspolymer, Acrylates/VA Copolymer, Acrylates/VP Copolymer, Butyl Ester of Ethylene/MA Copolymer, Butyl Ester of PVM/MA Copolymer, PVP, Shellac, Styrene/MA Copolymer, Polyacrylamide, and VP/VA Copolymer. Typically, the hair styling polymer is selected from acrylates copolymer, polyvinylprrolidone (PVP) or Acrylates/VA Copolymer, or alternatively, the hair styling polymer is an acrylates copolymer resin.

The hair conditioning agents useful as a hair care agent in the compositions of the present invention include cationic polymers, cationic surfactants, proteins, natural oils, hydrocarbons, quaternary ammonium functional silicones, silicone fluids, silicone emulsions, and mixtures thereof. Typically, the hair conditioning agent is selected from cationic polymers, cationic surfactants, or silicone emulsions, or alternatively, the hair conditioning agent is a silicone emulsion.

Various additional components useful in hair care compositions are described in U.S. Pat. No. 5,106,609 and U.S. Pat. No. 4,387,090; which are incorporated herein by reference in their entirety.

Emollients

Compositions according to the present invention optionally include emollients. Illustrative emollients, which are not limiting of the present invention, would include guerbet alcohols (such as isocetyl alcohol or isostearyl alcohol); esters (such as isopropyl palmitate, isopropyl isostearate, octyl stearate, hexyl laurate and isostearyl lactate); a liquid mixture of hydrocarbons which are liquids at ambient temperatures (such as petroleum distillates and light mineral oils); ethanol; volatile and non-volatile silicone oils, highly branched hydrocarbons, and non-polar carboxylic acids. The emollients can be included in the compositions of the present invention in amounts within the range of 0.01-70%, preferably 0.1-25%, by weight, of the total weight of the composition.

Surfactants and Emulsifiers

A variety of anionic, cationic, nonionic, and amphoteric surfactants can be used in the hair and skin care compositions of the present invention. Surfactants are used for cleaning, as well as emulsifiers in cosmetics. The functions of surfactants are to wet surfaces, and emulsify or solubilize oils. Representative, non-limiting examples include alkyl sulfates, alkyl ether sulfates, alkyl sulfonates, succinates, olefin sulfonates having about 12 to about 24 carbon atoms, ethoxylated alcohols, esters of sorbitol, esters of propylene glycol, and fatty acid esters of polyethylene glycol.

Useful surfactant examples can be found in Cosmetics and Toiletries Surfactant Encyclopedia, 2nd. Ed., by M. M. Rieger, and McCutcheon's, Detergents and Emulsifiers, North American Edition (2001), which are hereby incorporated by reference. The surfactants or emulsifiers can be included in the compositions of the present invention in amounts within the range of 0.01-30%, preferably 0.1-10%, by weight, of the total weight of the composition.

The surfactant can also be selected from silicone copolyols, or alternatively dimethicone copolyols. Representative examples include PEG/PPG-6/11 Dimethicone, PEG/PPG-8/14 Dimethicone, PEG/PPG-14/4 Dimethicone, PEG/PPG-17/18 Dimethicone, PEG/PPG-18/18 Dimethicone, PEG/PPG-20/6 Dimethicone, PEG/PPG-20/15 Dimethicone, PEG/PPG-20/20 Dimethicone, PEG/PPG-20/29 Dimethicone, PEG/PPG-25/25 Dimethicone, Dimethicone PEG-7 Phosphate, Dimethicone PEG-10 Phosphate, Dimethicone PEG/PPG-7/4 Phosphate, Dimethicone PEG/PPG-12/4 Phosphate, and Dimethicone PEG-7 Undecylenate.

Waxes

Waxes can be incorporated into the hair and skin care compositions of the present invention. Typically, waxes are used in such formulations to create solid or stick compositions such as lipsticks. The waxes can be selected from organic or silicone waxes. Representative non-limiting examples of hydrocarbon waxes include: Beeswax, Ceresin, Cetyl Esters, Citrus Aurantium Dulcis (Orange) Peel Wax, Copernicia Cerifera (Carnauba) Wax, Eclipta Prostrata Wax, Euphorbia Cerifera (Candelilla) Wax, Hydrogenated Japan Wax, Hydrogenated Jojoba Oil, Hydrogenated Microcrystalline Wax, Hydrolyzed Beeswax, Hydrolyzed Candelilla Wax, Hydrolyzed Carnauba Wax, Jojoba Esters, Lanolin Wax, Microcrystalline Wax, Mink Wax, Oxidized Beeswax, Oxidized Microcrystalline Wax, Ozokerite, Palm Kernel Wax, Paraffin, PEG-6 Beeswax, PEG-20 Beeswax, PEG-12 Carnauba, Persea Gratissima (Avocado) Wax, Potassium Oxidized Microcrystalline Wax, Shellac Wax, Simmondsia Chinensis (Jojoba) Butter, Simmondsia Chinensis (Jojoba) Seed Wax, Sulfurized Jojoba Oil, Synthetic Beeswax, Synthetic Candelilla Wax, Synthetic Carnauba, Synthetic Jojoba Oil, and Synthetic Wax.

Representative non-limiting examples of silicone waxes include C20-24 Alkyl Dimethicone, C24-28 Alkyl Dimethicone, C30-45 Alkyl Dimethicone, C30-45 Alkyl Dimethicone/Polycyclohexene Oxide Crosspolymer, C20-24 Alkyl Methicone, C24-28 Alkyl Methicone, and C30-45 Alkyl Methicone, Bis-PEG-12 Dimethicone Beeswax, Bis-PEG-12 Dimethicone Candelillate, Dimethiconol Beeswax, Dimethiconol Behenate, Dimethiconol Candelillate, and Dimethiconol Carnaubate.

Humectants and Moisturizers

Humectants and moisturizers can be incorporated into the hair and skin care compositions of the present invention. Typically, humectants and moisturizers are used in such formulations to create liquid or cream compositions such as conditioners, lotions and moisturizers. Representative non-limiting examples of humectants and moisturizers include Acetyl Arginine, Agave Atrovirens Extract, Alcaligenes Polysaccharides, Algae Extract, Aloe Barbadensis Leaf Extract, Aloe Barbadensis Leaf Polysaccharides, Amidinoproline, Bacillus/Rice Bran Extract/Soybean Extract Ferment Filtrate, Betaine, Black Strap Powder, 2,3-Butanediol, Caprylyl Glycol/Glycerin/Polyacrylic Acid Copolymer, Chitosan Lauroyl Glycinate, Cholesterol/HMDI/Pullulan Copolymer, Coix Lacryma-Jobi (Job's Tears) Shell Extract, Coleus Forskohlii Root Extract, Diglycereth-7 Malate, Diglycerin, Diglycol Guanidine Succinate, Dipeptide-3, Dunaliella Bardawil Extract, Erythritol, Ethylhexyl Hydroxystearoyl Hydroxystearate, Fructose, Glucose, Glucosyl Hesperidin, Glucuronolactone, Glycereth-7 Glycolate, Glycerin, Glyceryl Dimaltodextrin, Glycol, Hesperetin Laurate, 1,2,6-Hexanetriol, Honey, Hydrogenated Honey, Hydrogenated Isocetyl Olivate, Hydrogenated Starch Hydrolysate, Hydrolyzed Wheat Protein/PEG-20 Acetate Copolymer, Hydroxypropyltrimonium Hyaluronate, Impatiens Balsamina Extract, Inositol, Isostearyl Acetyl Glutaminate, Lactic Acid, Lactitol, Lauramidobutyl Guanidine Acetate, Laur/Myrist/Palmitamidobutyl Guanidine Acetate, Lauryl Malamide, Maltitol, Maltose, Mannitol, Mannose, the series of Methoxy PEGs, Myristamidobutyl Guani dine Acetate, Myrist/Palmitamidobutyl Guanidine Acetate, Palmitamidobutyl Guanidine Acetate, The series of PEGs, PEG-15 Butanediol, PEG-3 Methyl Ether, PEG-4 Methyl Ether, PEG-5 Pentaerythrityl Ether, Polyglyceryl Sorbitol, Potassium Dextrin Octenylsuccinate, Potassium PCA, PPG-6-Sorbeth-245, PPG-6-Sorbeth-500, Propylene Glycol, Prunus Mume Fruit Extract, Pseudoalteromonas Ferment Extract, Rosa Canina Seed Extract, Saccharomyces/Prunus Extract Ferment Filtrate, Sea Water, Sodium Acetylated Hyaluronate, Sodium Dextrin Octenylsuccinate, Sodium Glucuronate, Sodium PCA, Sorbeth-6, Sorbeth-20, Sorbeth-30, Sorbeth-40, Sorbitol, Sorbityl Silanediol, Stearyl Acetyl Glutaminate, Stearyl Palmitate, Sucrose, TEA-Dextrin Octenylsuccinate, Trehalose, Triglycereth-7 Citrate, Trimethylamine Oxide, Trioxaundecanedioic Acid, Tripropylene Glycol, Urea, Urea-d-Glucuronic Acid, Xylitol, and Xylose.

Pigments

Pigments can be incorporated into the hair and skin care compositions of the present invention. Typically, pigments are used in such formulations to create liquid, cream or powder compositions such as color cosmetics. Representative non-limiting examples of pigments include Aluminum Powder, Chromium Oxides, CI 77000, Manganese Oxides, Titanium Dioxide, Ultramarines, and Iron Oxides.

Thickeners

Thickeners can be incorporated into the hair and skin care compositions of the present invention. Typically, thickeners are used in such formulations to create liquid or cream compositions such as moisturizers, shampoos, conditioners, and color cosmetics. Representative non-limiting examples of thickeners include, Carbomer, Sodium Carbomer, Carboxylic Copolymers, Hydroxyethylcellulose, Methylcellulose, Ethylcellulose, Guar Gum, Xanthan Gum, Cellulose Gum, Gelatin, Hectorite, and Pectin.

Fragrances

As used herein, a fragrance can be any composition or material added to a personal care composition for the purpose of improving the olfactory sensory benefits of the formulation or applied formulation. Thus, the fragrance can be any perfume composition or composition of essential oils commonly used by those skilled in the art to create perfumes and fragrance compositions.

Active and Additional Ingredients

A wide variety of additional components can be employed in the hair care and topical skin compositions herein. The compositions of the present invention, especially the topical skin care compositions, can comprise a safe and effective amount of a pharmaceutical active or other active ingredients. Some of the typical active ingredients used in products such as these are antiacne agents, anticaries agents, antidandruff agents, antipruritic drugs, antifungal agents, antimicrobial agents (antibacterial, antifungal, antiprotozoal and antiviral drugs), antioxidants, cosmetic biocides, external analgesics, oral care agents, oral care drugs, oxidizing agents, reducing agents, skin bleaching agents, skin protectants, sunscreen agents, UV light absorbing agents, enzymes, surfactants, vitamins, fragrances, sunscreens, and insect repellents.

Some representative and non-limiting examples of antiacne agents are Salicylic acid and Sulfur. Some examples of anticaries agents are Sodium Fluoride, Sodium Monofluorophosphate, and Stannous Fluoride. Some examples of antidandruff agents are Coal tar, Salicylic acid, Selenium Sulfide, Sulfur, and Zinc Pyrithione. Some examples of antifungal agents are Calcium Undecylenate, Undecylenic Acid, Zinc Undecylenate, and Povidone-lodine. Some examples of antimicrobial agents are Alcohol, Benzalkonium Chloride, Benzethonium Chloride, Hydrogen Peroxide, Methylbenzethonium Chloride, Phenol, Poloxamer 188, and Povidone-Iodine.

Some representative and non-limiting examples of antioxidants are Acetyl Cysteine, Arbutin, Ascorbic Acid, Ascorbic Acid Polypeptide, Ascorbyl Dipalmitate, Ascorbyl Methylsilanol Pectinate, Ascorbyl Palmitate, Ascorbyl Stearate, BHA, p-Hydroxyanisole, BHT, t-Butyl Hydroquinone, Caffeic Acid, Camellia Sinensis Oil, Chitosan Ascorbate, Chitosan Glycolate, Chitosan Salicylate, Chlorogenic Acids, Cysteine, Cysteine HCI, Decyl Mercaptomethylimidazole, Erythorbic Acid, Diamylhydroquinone, Di-t-Butylhydroquinone, Dicetyl Thiodipropionate, Dicyclopentadiene/t-Butylcresol Copolymer, Digalloyl Trioleate, Dilauryl Thiodipropionate, Dimyristyl Thiodipropionate, Dioleyl Tocopheryl Methylsilanol, Isoquercitrin, Diosmine, Disodium Ascorbyl Sulfate, Disodium Rutinyl Disulfate, Distearyl Thiodipropionate, Ditridecyl Thiodipropionate, Dodecyl Gallate, Ethyl Ferulate, Ferulic Acid, Hydroquinone, Hydroxylamine HCl, Hydroxylamine Sulfate, lsooctyl Thioglycolate, Kojic Acid, Madecassicoside, Magnesium Ascorbate, Magnesium Ascorbyl Phosphate, Melatonin, Methoxy-PEG-7 Rutinyl Succinate, Methylene Di-t-Butylcresol, Methylsilanol Ascorbate, Nordihydroguaiaretic Acid, Octyl Gallate, Phenylthioglycolic Acid, Phloroglucinol, Potassium Ascorbyl Tocopheryl Phosphate, Thiodiglycolamide, Potassium Sulfite, Propyl Gallate, Rosmarinic Acid, Rutin, Sodium Ascorbate, Sodium Ascorbyl/Cholesteryl Phosphate, Sodium Bisulfite, Sodium Erythorbate, Sodium Metabisulfide, Sodium Sulfite, Sodium Thioglycolate, Sorbityl Furfural, Tea Tree (Melaleuca Aftemifolia) Oil, Tocopheryl Acetate, Tetrahexyldecyl Ascorbate, Tetrahydrodiferuloylmethane, Tocopheryl Linoleate/Oleate, Thiodiglycol, Tocopheryl Succinate, Thiodiglycolic Acid, Thioglycolic Acid, Thiolactic Acid, Thiosalicylic Acid, Thiotaurine, Retinol, Tocophereth-5, Tocophereth-10, Tocophereth-12, Tocophereth-18, Tocophereth-50, Tocopherol, Tocophersolan, Tocopheryl Linoleate, Tocopheryl Nicotinate, Tocoquinone, o-Tolyl Biguamide, Tris(Nonylphenyl) Phosphite, Ubiquinone, and Zinc Dibutyldithiocarbamate.

Some representative and non-limiting examples of cosmetic biocides are Aluminum Phenolsulfonate, Ammonium Phenolsulfonate, Bakuchiol, Benzalkonium Bromide, Benzalkonium Cetyl Phosphate, Benzalkonium Chloride, Benzalkonium Saccharinate, Benzethonium Chloride, Potassium Phenoxide, Benzoxiquine, Benzoxonium Chloride, Bispyrithione, Boric Acid, Bromochlorophene, Camphor Benzalkonium Methosulfate, Captan, Cetalkonium Chloride, Cetearalkonium Bromide, Cetethyldimonium Bromide, Cetrimonium Bromide, Cetrimonium Chloride, Cetrimonium Methosulfate, Cetrimonium Saccharinate, Cetrimonium Tosylate, Cetylpyridinium Chloride, Chloramine T, Chlorhexidine, Chlorhexidine Diacetate, Chlorhexidine Digluconate, Chlorhexidine Dihydrochloride, p-Chloro-m-Cresol, Chlorophene, p-Chlorophenol, Chlorothymol, Chloroxylenol, Chlorphenesin, Ciclopirox Olamine, Climbazole, Cloflucarban, Clotrimazole, Coal Tar, Colloidal Sulfur, o-Cymen-5-ol, Dequalinium Acetate, Dequalinium Chloride, Dibromopropamidine Diisethionate, Dichlorobenzyl Alcohol, Dichlorophene, Dichlorophenyl Imidazoldioxolan, Dichloro-m-Xylenol, Diiodomethyltolylsulfone, Dimethylol Ethylene Thiourea, Diphenylmethyl Piperazinylbenzimidazole, Domiphen Bromide, 7-Ethylbicyclooxazolidine, Fluorosalan, Formaldehyde, Glutaral, Hexachlorophene, Hexamidine, Hexamidine Diisethionate, Hexamidine Diparaben, Hexamidine Paraben, Hexetidine, Hydrogen Peroxide, Hydroxymethyl Dioxoazabicyclooctane, Ichthammol, Isopropyl Cresol, Lapyrium Chloride, Lauralkonium Bromide, Lauralkonium Chloride, Laurtrimonium Bromide, Laurtrimonium Chloride, Laurtrimonium Trichlorophenoxide, Lauryl Isoquinolinium Bromide, Lauryl Isoquinolinium Saccharinate, Laurylpyridinium Chloride, Mercuric Oxide, Methenamine, Methenammonium Chloride, Methylbenzethonium Chloride, Myristalkonium Chloride, Myristalkonium Saccharinate, Myrtrimonium Bromide, Nonoxynol-9 Iodine, Nonoxynol-12 Iodine, Olealkonium Chloride, Oxyquinoline, Oxyquinoline Benzoate, Oxyquinoline Sulfate, PEG-2 Coco-Benzonium Chloride, PEG-10 Coco-Benzonium Chloride, PEG-6 Undecylenate, PEG-8 Undecylenate, Phenol, o-Phenylphenol, Phenyl Salicylate, Piroctone Olamine, Sulfosuccinylundecylenate, Potassium o-Phenylphenate, Potassium Salicylate, Potassium Troclosene, Propionic Acid, PVP-Iodine, Quaternium-8, Quaternium-14, Quaternium-24, Sodium Phenolsulfonate, Sodium Phenoxide, Sodium o-Phenylphenate, Sodium Shale Oil Sulfonate, Sodium Usnate, Thiabendazole, 2,2′-Thiobis(4-Chlorophenol), Thiram, Triacetin, Triclocarban, Triclosan, Trioctyldodecyl Borate, Undecylenamidopropylamine Oxide, Undecyleneth-6, Undecylenic Acid, Zinc Acetate, Zinc Aspartate, Zinc Borate, Zinc Chloride, Zinc Citrate, Zinc Cysteinate, Zinc Dibutyldithiocarbamate, Zinc Gluconate, Zinc Glutamate, Zinc Lactate, Zinc Phenolsulfonate, Zinc Pyrithione, Zinc Sulfate, and Zinc Undecylenate.

Some representative and non-limiting examples of external analgesics are Benzyl Alcohol, Capsicum Oleoresin (Capsicum Frutescens Oleoresin), Methyl Salicylate, Camphor, Phenol, Capsaicin, Juniper Tar (Juniperus Oxycedrus Tar), Phenolate Sodium (Sodium Phenoxide), Capsicum (Capsicum Frutescens), Menthol, Resorcinol, Methyl Nicotinate, and Turpentine Oil (Turpentine).

Some representative and non-limiting examples of oral care agents are Aluminum Fluoride, Dicalcium Phosphate Dihydrate, Sodium Bicarbonate, Ammonium Fluoride, Domiphen Bromide, Sodium Chloride, Ammonium Fluorosilicate, Ferric Glycerophosphate, Sodium Fluoride, Ammonium Monofluorophosphate, Glycerin, Sodium Fluorosilicate, Ammonium Phosphate, Hexetidine, Sodium Glycerophosphate, Calcium Carbonate, Hydrated Silica, Sodium Metaphosphate, Calcium Fluoride, Hydrogenated Starch Hydrolysate, Sodium Monofluorophosphate, Calcium Glycerophosphate, Hydrogen Peroxide, Sodium Phytate, Calcium Monofluorophosphate, Hydroxyapatite, Sodium Styrene/Acrylates/Divinylbenzene, Calcium Phosphate, Magnesium Fluoride, Calcium Pyrophosphate, Magnesium Fluorosilicate, Stannous Fluoride, Cetylamine Hydrofluoride, Magnesium Glycerophosphate, Stannous Pyrophosphate, Cetylpyridinium Chloride, Manganese Glycerophosphate, Strontium Acetate, Chlorohexidine, Olaflur, Strontium Chloride, Chlorohexidine Diacetate, Phytic Acid, Tetrapotassium Pyrophosphate, Chlorohexidine Digluconate, Polyethylene, Tetrasodium Pyrophosphate, Chlorohexidine Dihydrochloride, Potassium Fluoride, Tricalcium Phosphate, Chlorothymol, Potassium Fluorosilicate, Zinc Chloride, Dequalinium Chloride, Potassium Glycerophosphate, Zinc Citrate, Diammonium Phosphate, Potassium Monofluorophosphate, Zinc Sulfate, and Dicalcium Phosphate.

Some representative and non-limiting examples of oral care drugs are Ammonium Alum, Potassium Alum, Benzyl Alcohol, Carbamide Peroxide, Elm Bark Extract, Gelatin, Glycerin, Hydrogen Peroxide, Menthol, Pectin, Phenol, Sodium Bicarbonate, Sodium Perborate, and Zinc Chloride.

Some representative and non-limiting examples of oxidizing agents are Ammonium Persulfate, Calcium Peroxide, Hydrogen Peroxide, Magnesium Peroxide, Melamine Peroxide, Potassium Bromate, Potassium Caroate, Potassium Chlorate, Potassium Persulfate, Sodium Bromate, Sodium Carbonate Peroxide, Sodium Chlorate, Sodium Iodate, Sodium Perborate, Sodium Persulfate, Strontium Dioxide, Strontium Peroxide, Urea Peroxide, and Zinc Peroxide.

Some representative and non-limiting examples of reducing agents are Ammonium Bisufite, Ammonium Sulfite, Ammonium Thioglycolate, Ammonium Thiolactate, Cystemaine HCl, Cystein, Cysteine HCl, Ethanolamine Thioglycolate, Glutathione, Glyceryl Thioglycolate, Glyceryl Thioproprionate, Hydroquinone, p-Hydroxyanisole, Isooctyl Thioglycolate, Magnesium Thioglycolate, Mercaptopropionic Acid, Potassium Metabisulfite, Potassium Sulfite, Potassium Thioglycolate, Sodium Bisulfite, Sodium Hydrosulfite, Sodium Hydroxymethane Sulfonate, Sodium Metabisulfite, Sodium Sulfite, Sodium Thioglycolate, Strontium Thioglycolate, Superoxide Dismutase, Thioglycerin, Thioglycolic Acid, Thiolactic Acid, Thiosalicylic Acid, and Zinc Formaldehyde Sulfoxylate.

Some representative and non-limiting examples of skin bleaching (or lightening) agents are Hydroquinone, ascorbic Acid, Kojic Acid and Sodium Metabisulfite.

Some representative and non-limiting examples of skin protectants are Allantoin, Aluminum Acetate, Aluminum Hydroxide, Aluminum Sulfate, Calamine, Cocoa Butter, Cod Liver Oil, Colloidal Oatmeal, Dimethicone, Glycerin, Kaolin, Lanolin, Mineral Oil, Petrolatum, Shark Liver Oil, Sodium Bicarbonate, Talc, Witch Hazel, Zinc Acetate, Zinc Carbonate, and Zinc Oxide.

Also useful herein are sunscreening agents. A wide variety of sunscreening agents are described in Chapter VIII, pages 189 et seq., of Cosmetics Science and Technology. Some representative and non-limiting examples of sunscreen agents are Aminobenzoic Acid, Cinoxate, Diethanolamine Methoxycinnamate, Digalloyl Trioleate, Dioxybenzone, Ethyl 4-[bis(Hydroxypropyl)] Aminobenzoate, Glyceryl Aminobenzoate, Homosalate, Lawsone with Dihydroxyacetone, Menthyl Anthranilate, Octocrylene, Octyl Methoxycinnamate, Octyl Salicylate, Oxybenzone, Padimate O, Phenylbenzimidazole Sulfonic Acid, Red Petrolatum, Sulisobenzone, Titanium Dioxide, and Trolamine Salicylate.

Some representative and non-limiting examples of UV light absorbing agents are Acetaminosalol, Allatoin PABA, Benzalphthalide, Benzophenone, Benzophenone 1-12, 3-Benzylidene Camphor, Benzylidenecamphor Hydrolyzed Collagen Sulfonamide, Benzylidene Camphor Sulfonic Acid, Benzyl Salicylate, Bornelone, Bumetriozole, Butyl Methoxydibenzoylmethane, Butyl PABA, Ceria/Silica, Ceria/Silica Talc, Cinoxate, DEA-Methoxycinnamate, Dibenzoxazol Naphthalene, Di-t-Butyl Hydroxybenzylidene Camphor, Digalloyl Trioleate, Diisopropyl Methyl Cinnamate, Dimethyl PABA Ethyl Cetearyldimonium Tosylate, Dioctyl Butamido Triazone, Diphenyl Carbomethoxy Acetoxy Naphthopyran, Disodium Bisethylphenyl Tiamminotriazine Stilbenedisulfonate, Disodium Distyrylbiphenyl Triaminotriazine Stilbenedisulfonate, Disodium Distyrylbiphenyl Disulfonate, Drometrizole, Drometrizole Trisiloxane, Ethyl Dihydroxypropyl PABA, Ethyl Diisopropylcinnamate, Ethyl Methoxycinnamate, Ethyl PABA, Ethyl Urocanate, Etrocrylene Ferulic Acid, Glyceryl Octanoate Dimethoxycinnamate, Glyceryl PABA, Glycol Salicylate, Homosalate, Isoamyl p-Methoxycinnamate, Isopropylbenzyl Salicylate, Isopropyl Dibenzolylmethane, Isopropyl Methoxycinnamate, Menthyl Anthranilate, Menthyl Salicylate, 4-Methylbenzylidene, Camphor, Octocrylene, Octrizole, Octyl Dimethyl PABA, Octyl Methoxycinnamate, Octyl Salicylate, Octyl Triazone, PABA, PEG-25 PABA, Pentyl Dimethyl PABA, Phenylbenzimidazole Sulfonic Acid, Polyacrylamidomethyl Benzylidene Camphor, Potassium Methoxycinnamate, Potassium Phenylbenzimidazole Sulfonate, Red Petrolatum, Sodium Phenylbenzimidazole Sulfonate, Sodium Urocanate, TEA-Phenylbenzimidazole Sulfonate, TEA-Salicylate, Terephthalylidene Dicamphor Sulfonic Acid, Titanium Dioxide, TriPABA Panthenol, Urocanic Acid, and VA/Crotonates/Methacryloxybenzophenone-1 Copolymer.

Some representative and non-limiting examples of insect repellents are benzamide NN-diethyl-3-methyl-m-delphene, NN-diethyl-m-toluamide, NN-diethyl-3-methylbenzamide (DEET), diethyl caprylamide and Ethyl Butylacetylaminopropionate.

Also useful in the present invention are sunless tanning agents, and anti-dandruff actives such as Zinc Pyrithione, Selenium Disulfide and Sulfur.

The present invention also provides a method for enhancing the fragrance longevity of a personal care composition comprising:

I) combining;

a) a fragrance

b) an organic solvent or silicone fluid,

c) a siloxane-based polyamide having the formula of claim 1 where the DP is greater than 30,

to form an oil phase,

(II) combining the oil phase with

d) a carrier optionally,

e) at least one personal care ingredient or active to form the personal care composition, and

(III) applying the personal care formulation to hair or skin to form a thin film.

The fragrance, organic solvent, silicone fluid, carrier, and optional personal care ingredient or active are any of those described supra and can be combined by any of the techniques described herein or others known in the art for preparing personal care formulations. The personal care formulations are applied by spreading the formulation on the surface of hair or skin to form a thin film.

### EXAMPLES

The following examples are presented to further illustrate the compositions and method of this invention, but are not to be construed as limiting the invention, which is delineated in the appended claims. All parts and percentages in the examples are on a weight basis and all measurements were obtained at about 23° C., unless indicated to the contrary.

The following examples illustrate hair and skin care compositions of silicone-polyamide copolymers (siloxane-based polyamides). The structural details of the silicone-polyamide copolymers used in these examples are summarized in Table 1 below. The silicone DP represents the average number of Si(CH₃)₂O units in each silicone block within the silicone-polyamide multi-block copolymer. The molecular weights of the silicone-polyamide copolymers were measured using Gel Permeation Chromatography.

The silicone-polyamide copolymers (“Copolymer”) used in these examples have units of the following formula:

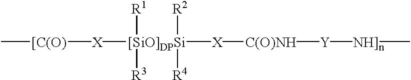

where R1, R2, R3 and R4 are methyl, X is (CH₂)₁₀ and Y is (CH₂)₆ and DP is defined in Table 1 and n is a value sufficient to give the molecular weight shown in Table 1.

TABLE 1
Silicone-polyamide Copolymers

| Copolymer # | #1 | #2 | #3 |
|---|---|---|---|
| Silicone DP | 15 | 30 | 100 |
| Molecular weight | 125,100 | 96,910 | 96,260 |

In the following, specific examples of hair care and skin care compositions within the scope of the present invention are also set forth. These specific hair and skin care examples are illustrative in connection with the present invention, and are not limiting. In the following, as well as throughout the present disclosure, names utilized are either Dow Corning Corporation product names, that is Dow Corning®, abbreviated DC® in the examples, or the CTFA (Cosmetics, Toiletry and Fragrance Association, Inc.) names, as set forth in the CTFA International Cosmetic Ingredient Dictionary (4th Ed. 1991). Throughout the description of this invention chemical abbreviations and symbols have their usual and customary meanings. While particular siloxane-based polyamides are disclosed or used in the following Examples, it is to be understood that other siloxane-based polyamides (for example, those made with a purified siloxane diacid, di-anhydride, diesters, or diacid chloride) may also be used and are within the spirit and scope of the invention.

### Example I

Hair Spray

Hair spray compositions were prepared from the following components utilizing conventional mixing techniques.

| | Weight % | |
|---|---|---|
| Ingredients | A | B |
| Deionized Water | 38.1 | — |
| Ethanol (SDA 40) | 1.0 | 47.5 |
| Isopropanol | — | 4.5 |
| Cyclomethicone (DC ® 245 fluid) | 54 | 38.1 |
| Silicone Copolyol (DC ® 193) | 0.5 | 0.5 |
| Triisopropanolamine | 0.4 | 0.4 |
| Copolymer #3 of Table 1 | 6.0 | — |
| Copolymer #1 of Table 1 | — | 3.0 |
| Acrylate Copolymers Resin (Gantrez ES 425) | | 6.0 |

Gantrez ES 425 is available from ISP Corporation, Wayne, NJ

These products are prepared by first dissolving the copolymer in the Cyclomethicone (A) or isopropanol (B) with stirring. The remaining ingredients are then added with stirring. The resulting hair spray compositions can then be packaged in a non-aerosol spray pump. Alternatively, the compositions can be combined with conventional propellants and packaged in an aerosol spray.

These hair sprays are useful for application to the hair to provide a styling and holding benefit, as well as sustained release of fragrance.

### Example II

Hair Conditioner

Hair conditioner compositions were prepared from the following components utilizing conventional mixing techniques.

| | Weight % | | | |
|---|---|---|---|---|
| Ingredient | A | B | C | Comp. |
| Phase 1 | | | | |
| Copolymer #2 from Table 1 | 0.5 | | | |
| Copolymer #3 from Table 1 | — | 0.5 | 0.5 | |
| Amino Emulsion (DC ® 929 Emulsion) | | | | 4 |
| Cyclomethicone (DC ® 245 Fluid) | 24.5 | 4.5 | — | |
| Dimethicone, 10 cSt. (DC ® 200 Fluid) | — | — | 24.5 | |
| Phase 2 | | | | |
| Deionized Water | 69.7 | 89.7 | 69.7 | 92.3 |
| Cetrimonium Chloride (Arquad 16-29) | 0.3 | 0.3 | 0.3 | |
| PEG 100 Stearate & Glyceryl Stearate | 1.0 | 1.0 | 1.0 | 1.0 |
| (Arlacel 165) | | | | |
| Cetearyl Alcohol (Lanette Wax O) | 2.5 | 2.5 | 2.5 | 1.0 |
| Hydroxyethyl Cellulose (Natrosol 250 | 1.50 | 1.5 | 1.5 | 1.5 |
| MR) | | | | |
| Preservative | | | | 0.2 |

Natrosol ™ 250 MR available from Hercules of Wilmington, DE
Lanette O available from Cognis Corp. of Hoboken, NJ
Arlacel ™ 165 available from Uniqema of Wilmington, DE
Arquad 16-29, 29% active, available from Akzo Nobel Chemicals, Dobbs Ferry, NY

Procedure:

Deionized water is added to the mixing vessel and heated to 75° C. With moderate agitation, disperse the Hydroxyethyl Cellulose until fully dissolved. Decrease heat to 60° C. and add Cetearyl Alcohol and Peg-100 Stearate & Glyceryl Stearate. Decrease heat to 40° C. and add Phase 1. Mix for 5-10 minutes and allow cooling to room temperature.

Slightly bleached European human hair from International Hair Importer and Products Inc was used for testing the conditioners in Example II. A master hand of hair about eight inches in length was subdivided into a series of individual hair tresses. Each tress weighed about 2.5 grams. A ½ inch of the root end of the hair was trimmed and glued to a 2″×2″ plastic tab using DUCO CEMENT™. The cement was allowed to dry, and the hair tress was combed and trimmed to a length, which allowed six inches of hair to extend below the bottom of the plastic tab. A hole was punched in middle of tab ˜¼″ from the top. Each tress was rinsed for 15 seconds under 40 C tap water. Using a pipette, 1.0 g of a 9% Sodium Lauryl Sulfate (active) solution was applied and lathered through the tress for 30 seconds. The tress was rinsed for 30 seconds under running water. Excess water was removed from the tress by passing the tress between the index and middle fingers. The tresses were placed on a tray covered with paper towels and dried overnight. Each tress was hand combed three times with the narrow teeth of an ACE™ comb and evaluated using the INSTRON “WET” and the INSTRON “DRY” COMBING procedures.

For tests involving the rinse-off conditioner, the hair tress is rinsed with tap water for 30 seconds at 40° C. The test conditioner is applied to the tress in the amount of 0.8 g and the tress is stroked for 30 seconds. The tress is rinsed for 30 seconds under tap water at 40° C. The excess water is removed by pulling the tress through the index and middle fingers. The tresses are allowed to dry separately on a paper towel, overnight at room temperature. The tresses are combed once before performing the Instron study.

INSTRON COMBING is an industry recognized test for determining hair conditioning by the ease of wet combing and the ease of dry combing. The test employs an INSTRON strain gauge that is equipped to measure the force required to comb the hair. Conditioning performance is based on the ability of a particular hair treatment formulation such as a shampoo or a hair conditioner to reduce the force required to comb the hair with the INSTRON strain gauge. The force is reported as Average Combing Load (ACL). The lower ACL value, the better the conditioning effect imparted by the formulation being tested. Typically, ACL baselines are initially established with “untreated” tresses that have only been washed with the Sodium Lauryl Sulfate solution. The effectiveness of a treatment can be expressed as the ACL of the treated tress or the % Reduction in ACL which is calculated by ((untreated hair ACL-treated hair ACL)/untreated hair ACL)*100.

According to the INSTRON WET COMBING method, the hair is first wet by dipping it in distilled water and then the hair is detangled by combing the tress three times. The tress is then retangled by dipping in distilled water three times. The excess water is removed by passing the tress through index and middle fingers twice. The tress is then placed on the hanger and INSTRON combed. The “retangle” and “Instron combing” steps are repeated until all data points are collected. An average combing force of three tresses is measured for each treatment.

According to the INSTRON DRY COMBING method, the hair is detangled by combing the tress 3 times. Then the hair is retangled by swirling the tress clockwise 3 times and counter-clockwise 3 times. The tress is then placed on the hanger and INSTRON combed. The “retangle” and “Instron combing” steps are repeated until all data points are collected. An average combing force of three tresses is measured for each treatment. The results of the INSTRON DRY COMBING test conducted with the conditioners of the present invention showed that Formulation C gave equivalent performance to a formulation containing an Amino Emulsion, used in the hair care industry as a conditioning silicone polymer. In addition, the formulations containing the silicone-polyamide copolymers gave hair tresses having good shine.

This product was useful as a rinse off hair conditioner and the silicone-polyamide provides thickening, a styling benefit, and sustained fragrance release.

### Example III

Rinse-Off Hair Conditioner

A hair conditioner composition can be prepared from the following components utilizing conventional mixing techniques.

| Ingredients | Weight % |
|---|---|
| Phase A | |
| Hydroxyethyl cellulose (Natrosol 250 | 1.5 |
| HHR) | |
| Deionised water | 50.0 |
| Phase B | |
| Cetrimonium Chloride (Arquad 16-29) | 0.3 |
| Phase C | |
| Cetyl Alcohol | 1.0 |
| Benzophenone 3 | 1.0 |
| #1 Copolymer from Table 1 | 2.0 |
| Phase D | |
| Glycerine | 1.0 |
| Deionised water | to 100 Wt. % |
| Preservative | q.s. |

Procedure:

1) Heat water of phase A to 70° C. and add the Natrosol under agitation.

2) Melt all the Phase C ingredient together at 100° C.

3) Add phase A while still hot to melted phase C very slowly.

4) Add phase C.

5) Cool at room temperature and add phase B ingredients.

6) Compensate the loss of water due to heating.

This application of the rinse-off conditioner containing the copolymer reduces the loss of hair colorant normally due to shampooing and exposure to UV light. In the chart below, 1 cycle corresponds to 7 hours UV exposure, one shampoo and rinsing off of shampoo composition plus brushing of the hair tresses. Lower Delta E numbers correspond to less color loss.

Delta E

| | Formulation | Control |
|---|---|---|
| 1 cycle | 0.8 | 1.9 |
| 2 cycles | 2.3 | 4.9 |
| 3 cycles | 4.0 | 5.8 |
| 4 cycles | 3.9 | 7.0 |
| 5 cycles | 3.8 | 7.0 |
| 6 cycles | 5.0 | 7.5 |
| 7 cycles | 4.0 | 7.8 |

### Example IV

Shampoo Composition

A shampoo composition can be prepared from the following components utilizing conventional mixing techniques.

| Ingredients | Weight % |
|---|---|
| Styling Agent | |
| Copolymers from Table | 1.00 |
| 1 | |
| Cyclomethicone | 9.0 |
| Premix | |
| Silicone emulsion | 1.0 |
| Main Mix | |
| Ammonium lauryl sulfate | 11.00 |
| Cocamide MEA | 2.0 |
| Cetyl alcohol | 1.0 |
| Dimethicone copolyol | 0.5 |
| Water | 74.5 |

This shampoo will be useful for cleansing the hair and for providing a styling benefit.

### Example V

Sunscreen Composition

An oil-in-water emulsion can be prepared by combining the following components utilizing conventional mixing techniques.

| Ingredients | Weight % |
|---|---|
| Phase A | |
| Water | 82.9 |
| Phase B | |
| Octyl Methoxycinnamate | 7.50 |
| Oxybenzone | 1.00 |
| Cetyl Palmitate | 0.75 |
| Dimethicone | 1.00 |
| Tocopherol Acetate | 0.10 |
| Copolymers from Table | 1.75 |
| 1 | |
| Phase C | |
| Water | 2.00 |
| Butylene Glycol | 2.00 |
| Phase E | |
| Cyclomethicone | 1.00 |

This emulsion will be useful for topical application to the skin to provide protection from the harmful effects of ultraviolet radiation. The silicone-polyamide copolymer will provide wash off resistance to the sunscreen composition.

### Example VI

Water-in-Silicone Moisturizer

Leave-on moisturizers comprising a water-in-silicone emulsion were prepared by combining the following components utilizing conventional mixing techniques.

| Formulation | A | B | C | D |
|---|---|---|---|---|
| Material | Weight % | Weight % | Weight % | Weight % |
| Phase 1 | | | | |
| Dimethicone Copolyol | 10.0 | 10.0 | 10.0 | 10.0 |
| 10% active in cyclo- | | | | |
| methicone) | | | | |
| (DC ® 5225C | | | | |
| Surfactant) | | | | |
| Cyclomethicone | 10.0 | 10.0 | 10.0 | 10.0 |
| (DC ® 245 Fluid) | | | | |
| Copolymer #2 from | | 1.0 | | 2.0 |
| Table 1 | | | | |
| Copolymer #3 from | | | 1.0 | |
| Table 1 | | | | |
| Fragrance (Powder | 1.0 | 1.0 | 1.0 | 1.0 |
| Fresh) | | | | |
| Phase 2 | | | | |
| Water | 73.0 | 73.0 | 73.0 | 73.0 |
| Glycerin | 5.0 | 5.0 | 5.0 | 5.0 |
| Sodium Chloride | 1.0 | — | — | — |

Combine Phase 1 materials and heat at 50° C. with dual blade mixing at 800 rpm until Copolymer is well dispersed. Combine Phase 2 materials in a separate container. With mixing, add phase 2 over 10 minutes at 1376 rpm. Mix for an additional 10 minutes at 1375 rpm.

This emulsion is useful for application to the skin as a moisturizer. The silicone-polyamide provides thickening, durability and longer lasting fragrance release to the moisturizer.

Fragrance testing was performed by a trained sensory panel using the following procedure: To test sustained fragrance release from copolymer-containing samples, the same three formulations used for sensory testing were prepare with the addition of 1% “Powder Fresh” fragrance oil obtained from a major fragrance house. A film (approximately 0.05 g) was introduced into the bottoms of 2 inch diameter polystyrene Petri dishes over an area somewhat smaller than 2″ diameter (to approximate the dose over a skin site). Dishes were left open for 15 hr, 10 hr, and 3 hours. Dishes were closed 30-60 minutes prior to panelist evaluation. Panelists were given sets of dishes for each time point, e.g. a 16 hour set. Each set contained the three different formulations and panelests ranked them by intensity of scent (four sets of three dishes).

Data were analyzed using Friedman's method for randomized blocks with significance determined at α=0.05 and α=0.1. If a significance was determined within the blocks, multiple comparison testing was carried out using a nonparametric method based on the Mann-Whitney statistic with significance again determined at α=0.05 and α=0.1.

Formulation D gave significantly higher fragrance intensity at α=0.05 after 15 hours compared with the control.

In a paired comparison analysis, the binomial distributions for formulation C at the 10 hour time point was greater than 0.9, meaning that 75% of the panelists ranked formulation C higher in fragrance intensity after 10 hours compared with the control. Formulation B gave higher fragrance intensity after 15 hours compared with the control based on paired comparison analysis.

### Example VII

Oil-in-Water Moisturizer

Leave-on moisturizers comprising an organic oil-in-water emulsion were prepared by combining the following components utilizing conventional mixing techniques.

| | | | | | |
|---|---|---|---|---|---|
| Formulation | A | B | C | D | E |
| Material | Weight % | Weight % | Weight % | Weight % | Weight % |

Phase 1

| | | | | | |
|---|---|---|---|---|---|
| Polyoxyethylene (2) | 2.0 | 2.0 | 2.0 | 2.0 | 2.0 |
| stearyl ether | | | | | |
| Isododecane | 10.0 | 10.0 | 10.0 | 10.0 | 10.0 |
| Copolymer #2 from | — | 2.0 | 3.0 | — | — |
| Table 1 | | | | | |
| Copolymer #3 from | — | — | — | 2.0 | 3.0 |
| Table 1 | | | | | |
| Fragrance (Powder | 1.0 | 1.0 | 1.0 | 1.0 | 1.0 |
| Fresh) | | | | | |
| Phase 2 | | | | | |
| Water | 78.5 | 76.5 | 75.5 | 76.5 | 75.5 |
| Carbomer (1% active in | 8.5 | 8.5 | 8.5 | 8.5 | 8.5 |
| deionized water) | | | | | |

Combine Phase 1 materials and heat at 35-40° C. with dual blade mixing at 900 rpm until Copolymer is well dispersed. Combine Phase 2 materials in a separate container. With mixing, add phase 2 over 10 minutes at 1376 rpm. Mix for an additional 10 minutes at 1376 rpm.

This emulsion is useful for application to the skin as a moisturizer. The silicone-polyamide provides thickening, durability and longer lasting fragrance release to the moisturizer.

Fragrance testing was conducted by a trained sensory panel. The objective of this testing was to determine if any of the formulations containing the silicone-polyamide copolymers were different from the control in terms of their ability to retain fragrance (1% Powder Fresh, obtained from a major fragrance company).

Samples of 0.05 g of each formulation were prepared in 9×50 mm petri dishes. Prepared plates were exposed to ambient air for 1, 10 and 15 hours. At all other times until testing, the plates were covered and refrigerated. Samples, identified with three digit codes, were presented to the panelists in random order.

Panelists were asked to rank the samples in order of least fragrance to most fragrance. Data were analyzed using Friedman's method for randomized blocks with significance determined at α=0.05 and α=0.1. If a significance was determined within the blocks, multiple comparison testing was carried out using a nonparametric method with significance again determined at α=0.05 and α=0.1.

In two separate tests, formulations C, D and E all gave significantly higher fragrance intensity at α=0.05 after 1 hour compared with the control. Formulations C and D gave significantly higher fragrance intensity at α=0.05 after 15 hours compared with the control. In a paired comparison analysis, the binomial distribution for formulation E was greater than 0.9, meaning that 75% of the panelists ranked formulation E higher in fragrance intensity after 15 hours compared with the control.

Skinfeel Sensory Testing

Formulations A, B, C, D, and E were prepared without fragrance. The 1% fragrance was replaced with an additional 1% water in phase 2. Sensory testing for skin feel attributes was performed on formulations A, B, C, D, and E according to ASTM Standards E 1958-98 (Standard Guide for Sensory claim Substantiation) and E 253 (Terminology Relating to Sensory Evaluation of Materials and Products), and ISO Standard 6658 (Sensory Analysis-Methodology—General Guidance).

| | Formulation | | | | |
|---|---|---|---|---|---|
| Attribute | A | B | C | D | E |
| Wetness | 71 | 73 | 70 | 65 | 62 |
| Absorbency | 75 | 82 | 66 | 62 | 73 |
| Gloss | 27 | 27 | 26 | 24 | 21 |
| Slipperiness | 40 | 40 | 44 | 40 | 38 |
| Smoothness | 55 | 49 | 53 | 52 | 51 |
| Residue | 17 | 13 | 17 | 12 | 23 |
| Greasiness | 3 | 8 | 9 | 5 | 13 |
| Waxiness | 16 | 15 | 18 | 22 | 24 |
| Powdery feel | 14 | 8 | 10 | 10 | 10 |
| Silicone feel | 19 | 7 | 9 | 5 | 13 |
| (Immediate) | | | | | |
| Silicone feel | 19 | 11 | 12 | 4 | 9 |
| (After 5 min.) | | | | | |

Formulation D tended toward higher absorbency and waxiness, and lower silicone feel compared with the control. Formulation E tended toward lower wetness and higher residue, greasiness and waxiness than the control.

### Example VIII

Facial Moisturizer

A leave-on facial emulsion composition was prepared by combining the following components utilizing conventional mixing techniques.

| Formulation | A | B | C | D |
|---|---|---|---|---|
| Ingredient | Weight % | Weight % | Weight % | Weight % |
| Glycerin | 5.0 | 5.0 | 5.0 | 5.0 |
| Copolymer #2 from | 4.0 | — | 4.0 | — |
| Table 1 | | | | |
| Copolymer #3 from | — | 4.0 | — | 4.0 |
| Table 1 | | | | |
| Cyclomethicone | 36.0 | 36.0 | — | — |
| (DC ® 245 Fluid) | | | | |
| Dimethicone | — | — | 36.0 | 36.0 |
| (DC ® 200 fluid, 10 cs) | | | | |
| Water | 53.0 | 53.0 | 53.0 | 53.0 |
| Sepigel 305 | 2.0 | 2.0 | 2.9 | 2.0 |

Sepigel 305 is available from Seppic Inc., Fairfield, NJ.

This formulation is prepared by first dissolving the copolymer in the cyclomethicone with heating and stirring. The remaining ingredients are then added with stirring at 1200 rpm using a dual blade mixer.

This emulsion is useful for application to the skin as a moisturizer.

Sensory testing for skin feel attributes was performed on formulations A, B, C, and D according to ASTM Standards E 1958-98 (Standard Guide for Sensory claim Substantiation) and E 253 (Terminology Relating to Sensory Evaluation of Materials and Products), and ISO Standard 6658 (Sensory Analysis-Methodology—General Guidance). Attributes were evaluated immediately after rub-in. The formulations containing the silicone-polyamide copolymers gave higher absorbency, lower wetness, and were thicker than the formulation without silicone-polyamide copolymer.

### Example IX

Water-in-Silicone Skin Cream

A skin cream composition was prepared by combining the following components utilizing conventional mixing techniques.

| Formulation | A | B | C |
|---|---|---|---|
| Ingredient | Weight % | Weight % | Weight % |
| Phase A | | | |
| C12-15 Alkyl Benzoate | 2.5 | 2.5 | 2.5 |
| #1 Copolymer from Table | 2.0 | — | — |
| 1 | | | |
| Synthetic Beeswax | — | 2.0 | — |
| Cyclopentasiloxane (and) | 10.0 | 10.0 | 10.0 |
| PEG-12 Dimethicone | | | |
| Crosspolymer | | | |
| DC ® 9011 Silicone | | | |
| Elastomer | | | |
| Dimethicone and | 6.0 | 6.0 | 7.2 |
| Trisiloxane | | | |
| (DC ® 2-1184 Fluid) | | | |
| Phase B | | | |
| Deionized Water | 77.0 | 77.0 | 77.0 |
| Magnesium Sulfate | 1.0 | 1.0 | 1.0 |

Sensory testing for skin feel attributes was performed on formulations A, B, and C, according to ISO 5495-1983 “Sensory Analysis—Methodology—Paired Comparison” and a book by Meilgaard, Civille, and Carr, entitled ‘Sensory Evaluation Techniques, 3^{rd} Edition’, CRC Press, Boca Raton, 1999. Attributes were evaluated immediately after rub-in and again 5 minutes later. Formulation A gave significantly lower immediate oily feel and significantly higher immediate waxy feel at α=0.1 compared with the control formulation C. Formulation A also gave significantly higher 5 minute residue at α=0.05 compared with the control formulation C, indicating that the panelists felt more of a residual film after 5 minutes.

### Example X

Color Foundation
A color foundation composition containing a pigment blend was
prepared by combining the following components utilizing
conventional mixing techniques.

| Ingredients | Weight % |
|---|---|
| Phase 1 | |
| Pigment Blend (See below) | 32.9 |
| Dimethicone Copolyol (DC ® 5225C) | 6.8 |
| #3 Copolymer from Table 1 | 10.0 |
| Phase 2 | |
| Water | 49.2 |
| Sodium chloride | 0.9 |
| Polysorbate 20 (Tween 20) | 0.2 |

Tween ® 20 is a registered trademark of Imperical Chemical Industries PLC, London, England

Pigment Blend:

| Ingredient | Supplier | Weight % |
|---|---|---|
| Cyclomethicone | Dow Corning, Midland, MI | 50.00% |
| Titanium Dioxide | Cardre AS, South Plainfield, NJ | 13.16% |
| Red Iron Oxide | Cardre AS | 11.41% |
| Yellow Iron Dioxide | Cardre AS | 18.26% |
| Black Iron Oxide | Cardre AS | 7.17% |

Pigment Blend Procedure:

1. Place cyclomethicone into blender.

2. Add titanium dioxide and mix by pressing the pulse button for 2 seconds for 15 seconds total.

3. Add red pigment and mix with pulse button several times. Repeat with each pigment color.

4. When all materials have been dispersed, mix on high and shred for 30 seconds to grind the pigments.

5. Place premix into a round glass jar and place on a pail roller for 6 hours.

Foundation Procedure:

1. Combine ingredients in Phase A, mix until uniform using a dual blade, turbulent style mixing action.

2. Combine the ingredients in Phase B in separate beaker, mix until uniform using a magnetic stirrer.

3, Increase mixing speed of Phase A tp 1376 rpm and very slowly add Phase B. This addition should take 10 minutes.

4, Continue mixing for an additional 10 minutes.

This emulsion is useful for application to the skin as a color foundation. The silicone-polyamide copolymer #3 provided thickening and durability to the foundation, and should provide longer lasting fragrance release.

### Example XI

Water-in-Silicone Emulsion as Lotion

A skin lotion composition was prepared by combining the following components utilizing conventional mixing techniques.

| Ingredients | Wt. % | Trade Name/Supplier |
|---|---|---|
| Phase A: | | |
| Copolymer #1 from Table 1 | 2% | |
| C12-C15 Alkyl Benzoate | 4% | Crodamol ® AB/Croda, Inc., |
| | | New York, NY; |
| | | Finsolv ® TN/Finetex ®, Inc., |
| | | Elmwood Park, NJ |
| 2-1184 Fluid | 6% | Dow Corning |
| Phase B: | | |
| 9011 Silicone Elastomer | 10% | Dow Corning |
| Blend | | |
| Phase C: | | |
| Distilled Water | 77% | |
| Magnesium Sulfate | 1% | |

Procedure:

1. Heat Phase A to 100° C. with mixing to melt the silicone-polyamide copolymer.

2. Heat Phase C to 90° C. with mixing.

3. When the silicone-polyamide copolymer in Phase A is melted, add Phase B while maintaining the temperature at 85° C.

4. Begin turbulent mixing of the above mixture (Phase A and Phase B) using the dual blade configuration as shown in the Dow Corning Silicone Formulation Aid notebook.

5. Immediately after adding Phase B, add Phase C slowly maintaining temperature and turbulent mixing.

6. Remove mixture from heat and continue mixing while cooling to room temperature.

7. Pour into adequate containers.

### Example XII

Water-in-Silicone Emulsion as Rich Feel Night Cream

A skin cream composition was prepared by combining the following components utilizing conventional mixing techniques.

| Ingredient | Wt. % | Trade Name/Supplier |
|---|---|---|
| Phase A: | | |
| Copolymer #1 from Table 1 | 2% | |
| 9011 Silicone Elastomer | 10% | Dow Corning |
| Blend | | |
| PPG-3 Myristyl Ether | 13% | Croda, Inc. |
| 2-1184 Fluid | 7% | Dow Corning |
| Phase B: | | |
| Distilled Water | 67% | |
| Magnesium Sulfate | 1% | |

### Example XIII

Water-in-Silicone Emulsion as Skin Lotion or Cream

A skin care composition was prepared by combining the following components utilizing conventional mixing techniques.

| Ingredients | Wt. % | Trade Name/Supplier |
|---|---|---|
| Phase A: | | |
| Copolymer #1 from Table 1 | 2% | Dow Corning |
| 9011 Silicone Elastomer | 10% | Dow Corning |
| Blend | | |
| PPG-3 Myristyl Ether | 3% | Croda, Inc. |
| 2-1184 Fluid | 4% | Dow Corning |
| Phase B: | | |
| Distilled Water | 80% | |
| Magnesium Sulfate | 1% | |

Procedure:

1. Heat Phase A to 100° C. to melt the silicone-polyamide copolymer.

2. Cool down Phase A to 80° C.

3. Heat Phase B to 70° C.

4. Add Phase B to Phase A very slowly with turbulent mixing at 1300 rpm.

5. Use dual blade configuration as shown in the Dow Corning Silicone Formulation Aids notebook.

### Example XIV

Lipstick Composition

A lipstick composition was prepared by combining the following components utilizing conventional mixing techniques.

| Formulation | A | B | C |
|---|---|---|---|
| Ingredient | Weight % | Weight % | Weight % |
| Phase A | | | |
| White ozokerite wax | 4% | 4% | 4% |
| Cendellila wax | 11% | 11% | 11% |
| C-30 Alkylmethyl siloxane | 5% | 5% | 5% |
| DC ® AMS C-30 Wax | | | |
| Cyclopentasiloxane | 5% | — | — |
| DC ® 245 Fluid | | | |
| Oleyl alcohol (Novol) | 8% | 8% | 8% |
| Petrolatum | 4% | 4% | 4% |
| Lanolin oil | 9% | 9% | 9% |
| Avocado oil | 2% | 2% | 2% |
| Phase B | | | |
| Eutanol G | 25% | 25% | 25% |
| Copolymer #1 from Table 1 | — | 5% | — |
| Copolymer #3 from Table 1 | — | — | 5% |
| Phase C | | | |
| pigment blend | 27% | 27% | 27% |
| pigment blend | | | |
| Covasil TiO2 | 5.00% | 5.00% | 5.00% |
| Cyclopentasiloxane | 77.50% | 77.50% | 77.50% |
| Covasil red W3801 | 17.50% | 17.50% | 17.50% |

Novol is registered trademark of Croda Inc.
Eutanol G is a registered trademark of Cognis Corp., Cincinatti, OH
Covasil is a registered trademark of LCW, France

Procedure:

1. Melt phase A ingredients at 80° C.

2. Melt phase B ingredients at approximately 120° C.

3. Add phase B to phase A while heating.

4. Cool the mixture to approximately 80° C.

5. Add the pigment blend.

6. Mold the lipsticks and cool to approximately 5° C. for 1 hour.

Formulations B and C containing the Copolymers gave less transfer of lipstick at 95% confidence level (α=0.05) compared with formulation A. By comparison, an organic polyamide based lipstick was totally transparent but more yellow, much harder, and didi not have any pay out or shine on the skin compared to the formulation B containing Copolymer.

### Example XV

Transparent Lip Gloss

Clear lip-gloss compositions were prepared by combining the following components utilizing conventional mixing techniques.

| Ingredients | Wt. % | Wt. % | Trade Name/Supplier |
|---|---|---|---|
| Copolymer #1 from Table 1 | 16.5% | 16.5% | |
| 245 Fluid | 32.96% | 26% | Dow Corning |
| Isopropyl Myristate | 22.42% | 26% | |
| Caprylic/Capric Triglyceride | 22.42% | 26% | Crodamol GTCC/ |
| | | | Croda, Inc. |
| 554 Cosmetic Fluid | — | 5.5% | Dow Corning |
| 555 Cosmetic Fluid | 5.5% | — | Dow Corning |
| Reflecks ™ Really Rouge | 0.2% | — | Engelhard-CLAL LP, |
| | | | Carteret, NJ |

Procedure:

1. Weigh all the ingredients into a container.

2. Cover with aluminum fold to avoid evaporation while processing.

3. Heat to 100° C. with low shear mixing until all ingredients are melted.

4. Pour into lipstick mold that has been coated with 245 Fluid.

5. Put in the refrigerator at approximately 5° C. for 1 hour.

6. Remove lipstick from the mold.

The lipsticks made with the copolymer are as clear and rigid as a lipstick base containing an organic polyamide wax but with better pay out and shine on skin.

### Example XVI

Water-in-Oil Facial Moisturizer

A facial moisturizer composition was prepared by combining the following components utilizing conventional mixing techniques.

| Ingredients | Wt. % | Trade Name/Supplier Phase A: |
|---|---|---|
| DC ® 5200 Formulation | 2 g | Dow Corning |
| Aid | | |
| Blend A | 30 g | |
| Phase B: | | |
| NaCl | 1 g | |
| Glycerin | 3 g | |
| Distilled Water | up to 100% | |
| Blend A: | | |
| Crodamol GTCC | 45% | Croda, Inc. |
| DC ® 245 Fluid | 45% | Dow Corning |
| Copolymer #1 from | 3% | Dow Corning |
| Table 1 | | |

Procedure:

1. Prepare Blend A by mixing the different ingredients at 90° C.

2. Take the appropriate amount of Blend A (still at 90° C.) and add the DC 5200 under agitation.

3. Mix Phase B ingredients together and heat to 70° C.

4. Add Phase B ingredients very slowly to Phase A with strong agitation to minimize evaporation as much as possible.

5. When Phase B is completely added, keep mixing for an additional 5 minutes.

6. Let it cool down.

7. Pass through Silverson.

8. The quantity of glycerin could be increased to 10% if more adequate for the positioning.

### Example XVII

Silicone-in-Water Emulsion

A skin care emulsion composition was prepared by combining the following components utilizing conventional mixing techniques.

| Ingredients | Wt. % | Trade Name/Supplier |
|---|---|---|
| Phase A: | | |
| #1 Copolymer from Table 1 | 2% | |
| Crodafos CES | 4% | Croda, Inc. |
| C12-C15 Alkyl Benzoate | 4% | Croda, Inc. or Finetex ®, Inc. |
| DC ® 2-1184 Fluid | 6% | Dow Corning |
| Phase B: | | |
| Distilled Water | 84% | |

Procedure:

1. Heat Phase A to 100° C. to melt the silicone-polyamide copolymer.

2. Cool down Phase A to 80° C. to 85° C.

3. Heat Phase B to 80° C. to 85° C.

4. Add Phase A to Phase B with turbulent mixing at 800 rpm using a dual blade configuration as shown in the Dow Corning Silicone Formulation Aids notebook to generate turbulence.

5. Mix until the system cools down to room temperature.

### Example XVIII

Silicone-in-Water Emulsion

A skin care emulsion composition was prepared by combining the following components utilizing conventional mixing techniques.

| Ingredients | Wt. % | Trade Name/Supplier |
|---|---|---|
| Phase A: | | |
| Copolymer #1 from Table 1 | 2% | |
| Crodafos CES | 4% | Croda, Inc. |
| PPG-3 Myristyl Ether | 13% | Croda, Inc. |
| DC ® 2-1184 Fluid | 7% | Dow Corning |
| Phase B: | | |
| Distilled Water | 74% | |

Procedure:

1. Heat Phase A to 100° C. to melt the silicone-polyamide copolymer.

2. Cool down Phase A to 80° C. to 85° C.

3. Heat Phase B to 80° C. to 85° C.

4. Add Phase A to Phase B with turbulent mixing at 800 rpm.

5. Prefer to use a dual blade configuration as shown in the Dow Corning Silicone Formulation Aids notebook to generate turbulence.

6. Mix until the system cools down to room temperature.

### Example IXX

Water-in-Silicone-in-Water Multiple Emulsion as Skin Lotion or Cream

A skin care multiple emulsion composition was prepared by combining the following components utilizing conventional mixing techniques. Any water soluble active ingredient may be dissolved in the internal water phase.

| Ingredients | Wt. % | Trade Name/Supplier |
|---|---|---|
| Phase A: | | |
| Copolymer #1 from Table 1 | 2% | Dow Corning |
| DC ® 9011 Silicone Elastomer | 10% | Dow Corning |
| Blend | | |
| C12-C15 Alkyl Benzoate | 4% | Crodamol ® AB/Croda, Inc. |
| | | or Finsolv ® TN/Finetex ®, |
| | | Inc. |
| DC ® 2-1184 Fluid | 6% | Dow Corning |
| Phase B: | | |
| Distilled Water | 45% | |
| Magnesium Sulfate | 1% | |
| Phase C: | | |
| Rodosurf L790 | 3% | Rhodia Inc., Cranbury, NJ |
| Phase D: | | |
| Distilled Water | 29% | |

Procedure:

1. Heat Phase A to 100° C. to melt the silicone-polyamide copolymer.

2. Cool down Phase A to 85° C.

3. Heat Phase B to 80° C.

4. Add Phase B to Phase A very slowly with turbulent mixing at 1300 rpm, using dual blade configuration.

5. Continue mixing until mixture reaches room temperature.

6. Add Phase C with gentle mixing to incorporate the surfactant.

7. Add this mixture (Phases A, B and C) to Phase D very slowly with gentle mixing approximately 400 rpm.

### Example XX

Oil-in-Water-in-Oil Multiple Emulsion as Skin Cream or Lotion

A skin care multiple emulsion composition was prepared by combining the following components utilizing conventional mixing techniques. Any oil soluble active ingredient may be dissolved in the internal oil phase.

| Ingredients | Weight % | Weight % | Supplier |
|---|---|---|---|
| Phase A | | | |
| Polawax Emulsifying Wax NF | 2.0 | 2.0 | Croda Inc. |
| Mineral Oil | 5.0 | 3.0 | |
| Cetiol SN | 2.0 | 3.0 | Cognis |
| Phase B | | | |
| DI Water | 64.0 | 66.5 | |
| Glycerine | 5.0 | 3.0 | |
| Phase C | | | |
| Tween 20 | 2.0 | 2.5 | ICI |
| Phase D | | | |
| DC ® 9011 Silicone Elastomer | 10.0 | 10.0 | Dow Corning |
| Blend | | | |
| Copolymer #1 from Table 1 | 2.0 | 2.0 | |
| DC ® 1184 Fluid | 4.2 | 4.2 | Dow Corning |
| Crodamol AB | 3.8 | 3.8 | Croda Inc. |

Procedure:

1. Phase A: mix all ingredients and heat at 70° C. with mixing until a uniform blend is achieved.

2. Phase B: mix all ingreadients and heat at 70° C. mixing until a uniform blend is achieved.

3. Phase C: prepare phase C in a separate beaker.

4. Phase D: mix all ingredients and heat at 90° C. to melt the polyamide, then cool down to room temperature.

5. Add phase B into phase A and mix at 700 rpm, (the emulsion will invert). Continue mixing while cooling down to room temperature. Then add phase C to this emulsion with gentle stirring.

6. Add the previous blend to Phase D very slowly, mixing at 500 to 600 rpm until the particle size of the emulsion is unimodal. The particle size of this emulsion is about 1 micron.

### Example XXI

Suncare Stick

A stick composition incorporating sunscreen was prepared by combining the following components utilizing conventional mixing techniques.

| Formulation | A | B | C | D | E | F |
|---|---|---|---|---|---|---|
| Material | Weight % | Weight % | Weight % | Weight % | Weight % | Weight % |
| Copolymer #1 | 16.5 | 16.5 | 16.5 | 16.5 | 16.5 | 17.5 |
| from Table 1 | | | | | | |
| DC ® 245 Fluid | 26 | 33 | 26 | 33.1 | 33.1 | 37 |
| Isopropyl Myristate | 26 | 22.4 | 22.5 | 20.2 | 20.2 | 20 |
| Caprylic/Capric | 26 | 22.4 | 22.5 | 20.2 | 20.2 | 20 |
| Triglyceride | | | | | | |
| DC ® 554 Cosmetic | 5.5 | — | 5.5 | — | 5.5 | |
| Fluid | | | | | | |
| DC ® 555 Cosmetic | — | 5.5 | — | 5.5 | — | |
| Fluid | | | | | | |
| Pigments | — | 0.2 | — | — | — | |
| Parsol MCX | — | — | 7 | 4 | 4 | 5 |
| Fragrance- | — | — | — | 0.5 | 0.5 | 0.5 |
| Diamond Yellow | | | | | | |

Parsol MCX is a registered trademark of Hoffmann-LaRoche Inc., Parsippany, NJ
DC ® 554 Cosmetic Fluid and DC ® 555 Cosmetic Fluid are supplied by Dow Corning Corp.

Procedure:

1. Weigh all the ingredients into a container.

2. Cover with aluminum fold to avoid evaporation while processing.

3. Heat to 100° C. with low shear mixing until all ingredients are melted.

4. Pour into stick mold that has been coated with 245 Fluid.

5. Allow to harden at room temperature, or place mold in the refrigerator at approximately 5° C. for 1 hour.

All formulations gave solid sticks that ranged from transparent to opaque with appropriate stick hardness, good payout and shine on skin.

### Example XXII

Suncare and Insect Repellent Stick

A stick composition incorporating sunscreen and insect repellent was prepared by combining the following components utilizing conventional mixing techniques.

| Formulation | A | F | B | C | D | E | G |
|---|---|---|---|---|---|---|---|
| Material | Weight % | Weight % | Weight % | Weight % | Weight % | Weight % | Weight % |
| Copolymer #1 | 17 | 18 | 18 | 17 | 17 | 17 | 17 |
| from Table 1 | | | | | | | |
| DC ® 245 Fluid | 37.5 | 36.5 | 15.5 | 23.8 | 15.9 | 15.9 | — |
| Isopropyl | 15 | 10 | 15.5 | 23.7 | — | — | 23.75 |
| Myristate | | | | | | | |
| Caprylic/Capric | 15 | 10 | 15.5 | — | 31.6 | — | 23.75 |
| Triglyceride | | | | | | | |
| C12-15 Alkyl | — | — | — | — | — | 31.6 | — |
| Benzoate | | | | | | | |
| Parsol MCX | 5 | 5 | 5 | 5 | 5 | 5 | 5 |
| Fragrance- | 0.5 | 0.5 | 0.5 | 0.5 | 0.5 | 0.5 | 0.5 |
| Eucalyptol | | | | | | | |
| Dimethyl Pthalate | | | | | | | |
| Diethyl Tolumide | 10 | 20 | 30 | 30 | 30 | 30 | 30 |
| Ethyl Hexanediol | | | | | | | |
| (Unirep U-18) | | | | | | | |

Unirep U-18 is a registered trademark of Induchem AG, Switzerland

Procedure:

1. Weigh all the ingredients into a container.

2. Cover with aluminum fold to avoid evaporation while processing.

3. Heat to 100° C. with low shear mixing until all ingredients are melted.

4. Pour into stick mold that has been coated with 245 Fluid.

5. Allow to harden at room temperature, or place mold in the refrigerator at approximately 5° C. for 1 hour.

All formulations gave solid sticks that ranged from transparent to opaque with appropriate stick hardness and good payout.

### Example XXIII

Optimum use of the silicone-polyamide copolymers for easy addition into various emulsion formulations is achieved by first dissolving the solid copolymer in a mixture of silicone and organic solvents. When the copolymer/organic solvent/silicone solvent ratio is such that transparent, stable solutions are produced, the solutions may be added easily to emulsion, or other, formulations. When these clear, compatible solutions are used to make fully formulated hair and skin care products, the resulting hair and skin care products can be more stable, can be clear if desired, and have unique sensory skinfeel properties compared with mixtures of copolymer/organic solvent/silicone solvent that are hazy or incompatible.

Keeping Copolymer #1 from Table 1 constant at 5 weight % in the mixture of copolymer, silicone solvent and organic solvent, the following ratios of each component produces transparent, compatible mixtures. For example, in the case where a range of 20 to 45% D5 and 50 to 75% Crodamol GTCC is given, weight % ratios of 5 Copolymer /20 D5/75 Crodamol, 5 Copolymer/45 D5/50 Crodamol, and all ratios in between produced transparent solutions. Examples of a single ratio for an organic/silicone solvent combination are also given. These examples are not limiting and other ratios of the same solvent combinations may also produce transparent, compatible solutions. Copolymers #2 and #3 should exhibit the same behavior to produce transparent, compatible mixtures; however, the ratios of organic and silicone solvents may be different. In the following examples D5 is cyclopentasiloxane (DC® 245, Dow Corning Corporation, Midland, Mich.)

20-45% D5 and 50-75% Crodamol GTCC

55-60% D5 and 35-40% Finsolv TN

8.7-32% D5 and 64-87.3% PPG-3 Myristyl Ether

53% DC 2-1184 Fluid and 42% Finsolv TN

5-40% DC 2-1184 Fluid and 55-90% PPG-3 Myristyl Ether

Keeping the Copolymer #1 from Table 1 constant at 10 weight % in the mixture of copolymer, silicone solvent and organic solvent, the following ranges of silicone solvents and organic solvents produce transparent, compatible mixtures.

30-45% D5 and 45-60% Crodamol GTCC

5-20% D5 and 70-85% Crodamol ISNP

15-25% D5 and 65-75% Crodamol OS

55% D5 and 35% Finsolv TN

15-75% D5 and 15-75% Isostearyl Alcohol

45% D5 and 45% Mineral Oil

15-75% D5 and 15-75% Oleyl Alcohol

10-55% D5 and 35-80% PPG-3 Myristyl Ether

65% D5 and 25% Sunflower Seed Oil

15-60% DC 556 Fluid and 30-75% Isostearyl Alcohol

15-75% DC 556 Fluid and 15-75% Oleyl Alcohol

5-15% DC 2-1184 Fluid and 75-85% Crodamol ISPN

45% DC 2-1184 Fluid and 45% Crodamol LGE

15% DC 2-1184 Fluid and 75% Crodamol OS

50% DC 2-1184 Fluid and 40% Finsolv TN

20-85% DC 2-1184 Fluid and 5-70% Isostearyl Alcohol

15-75% DC 2-1184 Fluid and 15-75% Oleyl Alcohol

10-50% DC 2-1184 Fluid and 40-80% PPG-3 Myristyl Ether

20-30% 2-1184 Fluid and 60-70% Crodamol GTCC

5-10% 556 Fluid and 80-85% ISPN

40-50% 556 Fluid and 40-50% Isopar H

20-25% 2-5562 Fluid and 65-70% Crodamol GTCC

50% 2-5562 Fluid and 40% Finsolv TN

5-50% 2-5562 Fluid and 40-85% Isostearyl Alcohol

45% 2-5562 and 45% Oleyl Alcohol

5-20% 2-5562 Fluid and 70-85% PPG-3 Myristyl Ether

Keeping Copolymer #1 from Table 1 constant at 30 weight % in the mixture of copolymer, silicone solvent and organic solvent, the following ratios of each component produces transparent, compatible mixtures.

40% 200 Fluid, 5 cSt. and 30% Finsolv TN

25-55% D5 and 15-45% Crodamol GTCC

35% D5 and 35% Crodamol ISPN

35-46.6% D5 and 23.4-35% Finsolv TN

50% D5 and 20% PPG-3 Myristyl Ether

11.6-23.3% DC 2-1184 Fluid and 46.7-58.4% Crodamol OS

Keeping Copolymer #1 from Table 1 constant at 50 weight % in the mixture of copolymer, silicone solvent and organic solvent, the following ratios of each component produces transparent, compatible mixtures.

10-40% D5 and 10-40% Crodamol GTCC

35-40% D5 and 10-15% Finsolv TN

15-37.5% DC 2-1184 Fluid and 12.5-35% Finsolv TN

Finesolv TN is a registered trademark of Finetex Inc., Elmwood Park, N.J.

Isopar H is a registered trademark of ExxonMobil Chemicals, Houston, Tex.

Crodamol is a registered trademark of Croda, Inc., New York, N.Y.

We claim:

1. A base composition comprising:
A) a silicone fluid,
B) a silicone immiscible organic substance,
C) a siloxane-based polyamide having the formula; 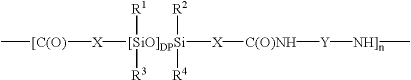
where,
(1) DP (degree of polymerization) is 1-700,
(2) n is 1-500,
(3) X is a linear or branched chain alkylene having 1-30 carbons,
(4) Y is a linear or branched chain alkylene having 1-40 carbons,
wherein,
(a) the alkylene group may optionally and additionally contain in the alkylene portion at least one of (i) 1-3 amide linkages; (ii) a C5 or C6 cycloalkane; or (iii) phenylene, optionally substituted by 1-3 members which are independently C1-C3 alkyls;
(b) the alkylene group itself may optionally be substituted by at least one of (i) hydroxy; (ii) a C3-C₈ cycloalkane; (iii) 1-3 members which are independently C1-C3 alkyls; phenyl, optionally substituted by 1-3 members which are independently C1-C3 alkyls; (iv) a C1-C3 alkyl hydroxy; or (v) a C1-C6 alkyl amine;
(c) Y can be Z where Z is T(R²⁰)(R²¹)(R²²) where R²⁰, R²¹ and R²² are each independently linear or branched C1-C10 alkylenes; and T is CR in which R is hydrogen, the group defined for R¹-R⁴, or a trivalent atom such as N, P and Al;
(5) each of R¹-R⁴ is independently methyl, ethyl, propyl, isopropyl, a siloxane chain, or phenyl, wherein the phenyl may optionally be substituted by 1-3 members, which are methyl or ethyl;
(6) X, Y, DP, and R¹-R⁴ may be the same or different for each polyamide unit, wherein the base composition is a stable dispersion.

2. The composition of claim 1 wherein the base composition is a clear solution.

3. The composition of claim 1 wherein the silicone fluid is a volatile methyl silicone.

4. The composition of claim 1 wherein the silicone fluid is a polydimethylsiloxane having a viscosity of 20 cs (mm/s²) or greater.

5. The base composition of claim 1 wherein the silicone fluid is a phenyl containing polysiloxane.

6. The composition of claim 1 wherein the silicone immiscible organic substance is an organic oil selected from esters, fatty alcohols, glycerides, hydrocarbons, and vegetable oils.

7. The composition of claim 1 wherein the silicone immiscible organic substance is a personal care ingredient selected from emollients, surfactants, emulsifiers, waxes, humectants, thickeners, and personal care actives.

8. The base composition of claim 1 further comprising
D) a silicone miscible solvent.

9. The base composition of claim 8 wherein the silicone miscible solvent is selected from volatile methyl siloxanes, low molecular weight alcohols, ethoxylated alcohols, propoxylated alcohols, and mixtures thereof.

10. The base composition of claim 1 further comprising a silicone elastomer.

11. The base composition of claim 1 wherein the components are present in the following weight percents:
A) 0.1 to 50%
B) 0.1 to 90%
C) 0.1 to 90%
with the proviso that the total of the components equal 100%.

12. The base composition of claim 1 wherein the DP of the siloxane-based polyamide is 30 or greater and further comprising a fragrance.

13. A hair care composition comprising:
a) the siloxane-based polyamide as described in claim 1,
b) an organic solvent or a silicone fluid
c) a hair care agent selected from hair styling polymers or hair conditioning agents
d) optionally, a silicone copolyol.

14. The hair care composition of claim 13 wherein the hair styling polymer is selected from Acrylates Copolymer, Polyvinylprrolidone (PVP) and Acrylates/VA Copolymer.

15. The hair care composition of claim 13 where the hair styling polymer is an acrylate copolymers resin.

16. The hair care composition of claim 13 where the hair conditioning agent is selected from cationic polymers, cationic surfactants, fatty alcohols, fatty esters, and silicone emulsions.

17. The hair care composition of claim 13 where the hair-conditioning agent is a silicone emulsion.

18. A skin care composition comprising;
a) the base composition of claim 1,
b) a hydrocarbon wax, and
c) a pigment.

19. The skin care composition of claim 18 further comprising a silicone wax.

20. A multiple phase emulsion comprising at least one oil phase containing the base composition of claim 1.

21. A water in silicone emulsion comprising at least one oil phase containing the base composition of claim 1.

22. A method for enhancing the fragrance longevity of a personal care composition comprising:
I) combining;
a) a fragrance
b) an organic solvent or silicone fluid,
c) a siloxane-based polyamide having the formula of claim 1 where the DP is 30 or greater
to form an oil phase,
(II) combining the oil phase with
d) a carrier optionally,
e) at least one personal care ingredient or active to form the personal care composition, and
(III) applying the personal care formulation to hair or skin to form a thin film.
